SEQUENCE LISTING (8 sequences)

SEQ ID NO: 1 - DNA, 1637 bp, synthetic construct
  source 1..1637: mol_type = other DNA
gtccctgtcgccggggctgatacagccacaacctgccgcactgactgtccagggtaaatgaacagcccacaaaattttttacccaaaacccggtaaaaaattttgcgttctgcccaggacaggtgtgtcaggccgtggcagtgatgccccttgcggcgaaggttctcaaaaaggtgatggtgaataccggagggaacaaaaggcaggtccccgcaaaacgtcaaaaccgcggctcccgaaactcaaaagatgatgggggctgaaaacatcaacagaaggagacacatcatgaacataaatgacttgatcagagagataaagaataaagactacactgtcaagttatcgggaacggacagcaattcgatcacccagttaataatccgggtaaataatgacggcaatgaatatgtcatttcagagtccgaaaatgaatccattgtcgaaaaattcatttcggcgttcaagaatggctggaatcaagaatacgaagatgaggaggaattttacaacgacatgcaaacaatcacactgaaaagtgaattaaattaactcggtaccaaattccagaaaagaggcctcccgaaaggggggccttttttcgttttggtccccgacgtctgtaatgtagcctaagccctatgcccatgatctactgacgtccaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatca

SEQ ID NO: 2 - DNA, 1718 bp, synthetic construct
  source 1..1718: mol_type = other DNA
aacgccgtttcgcaaaccgcgcttcacgtcgttgcggcgggtccctgctcattgcctgccgggtgccacacacccgtatgagtccctgtcgccggggctgatacagccacaacctgccgcactgactgtccagggtaaatgaacagcccacaaaattttttacccaaaacccggtaaaaaattttgcgttctgcccaggacaggtgtgtcaggccgtggcagtgatgccccttgcggcgaaggttctcaaaaaggtgatggtgaataccggagggaacaaaaggcaggtccccgcaaaacgtcaaaaccgcggctcccgaaactcaaaagatgatgggggctgaaaacatcaacagaaggagacacatcatgaacataaatgacttgatcagagagataaagaataaagactacactgtcaagttatcgggaacggacagcaattcgatcacccagttaataatccgggtaaataatgacggcaatgaatatgtcatttcagagtccgaaaatgaatccattgtcgaaaaattcatttcggcgttcaagaatggctggaatcaagaatacgaagatgaggaggaattttacaacgacatgcaaacaatcacactgaaaagtgaattaaattaactcggtaccaaattccagaaaagaggcctcccgaaaggggggccttttttcgttttggtccccgacgtctgtaatgtagcctaagccctatgcccatgatctactgacgtccaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatca

SEQ ID NO: 3 - DNA, 69 bp, synthetic construct
  source 1..69: mol_type = other DNA
tcttcctcttcttcctcttcttcctcttctttccgttcctctcctgcttaaacgccgtttcgcaaaccg

SEQ ID NO: 4 - DNA, 68 bp, synthetic construct
  source 1..68: mol_type = other DNA
tttcccaggcgtcctacgaggatgaccttgttaatgccacgaactgccattgatcggcacgtaagagg

SEQ ID NO: 5 - DNA, 68 bp, synthetic construct
  source 1..68: mol_type = other DNA
cttacctggtttcatcttcataccttattgggtgtattaaaaacagtagggtccctgtcgccggggct

SEQ ID NO: 6 - DNA, 75 bp, synthetic construct
  source 1..75: mol_type = other DNA
ctgatgttgagcagtcatggagaattgaaacttgtttaagactttggcagtgatcggcacgtaagaggttccaac

SEQ ID NO: 7 - DNA, 75 bp, synthetic construct
  source 1..75: mol_type = other DNA
tcttcctcttcttcctcttcttcctcttctttccgttcctctcctgcttatgatcggcacgtaagaggttccaac

SEQ ID NO: 8 - DNA, 68 bp, synthetic construct
  source 1..68: mol_type = other DNA
gatgaccttgttaatgccacgaactgccatgatgtgtctccttctgttgagtccctgtcgccggggct

(19) United States
(12) Patent Application Publication
(10) Pub. No.: US 2026/0218209 A1
(43) Pub. Date: Jul. 30, 2026

(54) METHODS AND CONSTRUCTS FOR IMPROVING CONJUGATION EFFICIENCY

(71) Applicant: RAMOT AT TEL AVIV UNIVERSITY LTD., Tel Aviv (IL)

(72) Inventors: Bruria SAMUEL, Tel Aviv (IL); David BURSTEIN, Tel Aviv (IL); Karin MITTELMAN, Tel Aviv (IL); Shirly Ynbal CROITORU, Tel Aviv (IL)

(21) Appl. No.: 19/145,233

(22) PCT Filed: Feb. 5, 2024
(86) PCT No.: PCT/IL2024/050132

§ 371 Date: Jul. 2, 2025

Related U.S. Application Data
(60) Provisional application No. 63/443,488, filed on Feb. 6, 2023.

(51) Int. Cl. C12N 15/70; C07K 14/255; C12N 9/10; C12N 9/22; C12N 9/90

(52) U.S. Cl. CPC C12N 15/70; C07K 14/255; C12N 9/1007; C12N 9/22; C12N 9/90

(57) ABSTRACT

The present invention is directed to a method for increasing conjugation efficiency of a basic conjugative element by inserting into the leading region sequence of the conjugative element an anti-defense nucleotide sequence encoding at least one anti-defense gene.

### FIELD OF THE INVENTION

The present disclosure is generally directed to the field of introduction of DNA into cells by conjugation. In particular, the invention is directed to improved constructs for use in conjugation by introducing anti-defense genes.

### BACKGROUND

Bacterial conjugation is a major horizontal gene transfer (HGT) mechanism in which DNA is transferred from a donor to a recipient cell by direct contact. It is an important driver of rapid bacterial evolution, promoting the acquisition of various genes, including metabolic pathways, virulence factors, and antimicrobial resistance genes. The DNA transport machinery of conjugative elements includes type IV secretion system (T4SS) proteins, an origin of transfer (oriT), and a DNA-processing nucleoprotein complex called the relaxosome, composed of the relaxase and often additional auxiliary proteins. While conjugative plasmids and integrative conjugative elements (ICEs) encode for the entire transport machinery, some mobile genetic elements (MGEs) contain only the relaxase gene and an oriT sequence but lack the machinery required for self-transfer. Such elements, termed “mobilizable plasmids”, can be transferred using the conjugation machinery of a co-residing conjugative element.

Conjugation initiation occurs when the relaxosome is assembled on the plasmid's oriT, and the relaxase nicks the nic site, located within the oriT. The nicked DNA strand is then transferred with the covalently attached relaxase through the T4SS into the recipient cell. The region first transferred to the recipient cell is defined as the leading region. The relaxase gene is typically located on the plasmid in close proximity to the oriT, in the lagging region, which is the last to enter the recipient cell.

Previous studies suggested that genes in the leading region are important for plasmid stability during conjugation. It was demonstrated that, in certain conjugative plasmids, leading region genes are expressed early upon the entry of the plasmid into the recipient cell, even before the transfer is complete. The leading regions of these plasmids contain promoters designated Frpo (ssiD and ssiE in F plasmid, and ssi2 and ssi3 in the IncI1 plasmid Col1b-9). These promoters can adopt a stem-loop structure such that the regions of the −10 and −35 elements mimic a double-strand conformation, allowing their recognition by the host RNA polymerase. It was therefore proposed that Frpo may serve as a single-stranded promoter that allows early expression of the leading region genes. Indeed, the transcription levels and gene product accumulation indicate rapid expression of genes located in the leading region.

Similar to other foreign genetic material, conjugative elements face a wide repertoire of prokaryotic defense systems, including restriction-modification (R-M), clustered regularly interspaced short palindromic repeats (CRISPR)-Cas, and more. Despite these defense systems, designed to prevent the entry of exogenous DNA, HGT widely persists across species. This is enabled owing to different mechanisms mobile genetic elements have developed to overcome prokaryotic defense systems, including the expression of anti-defense proteins such as anti-restriction and anti-CRISPR proteins.

Except for a few examples of anti-defense genes (ArdA and PsiB), little is known about the annotation and function of genes in the leading regions of conjugative elements and their function during conjugation.

Conjugative elements, such as plasmids, have been tested as potential conjugative delivery systems for various biotechnological applications, such as targeting antibiotic-resistance bacteria using CRISPR nucleases. However, these attempts resulted in low conjugation efficiency, especially in complex microbial communities like the human gut. These studies emphasize that improving conjugation efficiency is vital for future applications.

Accordingly, there is a need in the art for methods and compositions for improving conjugation efficiency, based on the use of anti-defense genes.

### SUMMARY OF THE INVENTION

The following embodiments and aspects thereof are described and illustrated in conjunction with compositions and methods which are meant to be exemplary and illustrative, not limiting in scope. In various embodiments, one or more problems have been reduced or eliminated, while other embodiments are directed to other advantages or improvements.

The inventors have shown in the present invention that anti-defense genes are generally concentrated in anti-defense islands located in the leading region of natural conjugative elements and are encoded on the strand complementary to the T-strand (herein defined as T-strand complement). As a result, they are transcribed from the single strand first entering the cell in an early stage of the transfer. This early expression of the anti-defense genes appears to protect the transferred DNA and facilitate higher conjugation efficiency when defense systems are present in the recipient. This makes the anti-defense islands excellent candidates for adding to existing conjugative elements in order to improve conjugation efficiency.

In some embodiments, the present invention provides a method for increasing conjugation efficiency of a basic conjugative element which comprises an origin of transfer (oriT) sequence defining a leading region sequence, a T-strand, and a T-strand complement, the method comprising inserting into the leading region sequence an anti-defense nucleotide sequence comprising a sequence encoding at least one anti-defense gene, thereby generating an improved conjugative element, wherein: the at least one anti-defense gene is inserted within about 35 kb from the oriT and/or within the about 30 open reading frames (ORFs) closest to the oriT such that it is encoded on the T-strand complement; and the conjugation efficiency of the improved conjugative element is increased compared to the conjugation efficiency of the basic conjugative element.

In some embodiments, the at least one anti-defense gene is an anti-CRISPR gene; an anti-restriction gene, and/or an SOS inhibitor gene. In some embodiments, the anti-CRISPR (acr) gene is selected from acrIIA4, acrIC6, acrIF16, acrIE9, acrIB, and acrIIA21; the anti-restriction gene is selected from ocr, ardA, ardB, klcAHS, and darB; and/or the SOS inhibitor is selected from psiA and psiB. In some embodiments, the at least one anti-defense gene is at least two, at least three, at least four, or at least five anti-defense genes.

In some embodiments, the anti-defense nucleotide sequence further comprises a sequence encoding at least one anti-defense-related gene, and the at least one anti-defense-related gene is inserted within about 35 kb from the oriT and/or within the about 30 open reading frames (ORFs) closest to the oriT such that it is encoded on the T-strand complement. In some embodiments, the at least one anti-defense-related gene encodes a methyltransferase, a single-strand DNA-binding protein (SSB), a toxin, and/or an antitoxin. In some embodiments, the at least one anti-defense-related gene is at least two, at least three, at least four, or at least five anti-defense-related genes.

In some embodiments, the anti-defense nucleotide sequence further comprises a sequence encoding at least one umu gene or homolog thereof, which is positioned farther away from the oriT compared to the at least one defense gene. In some embodiments, the at least one umu gene is a homolog of umuC and/or a homolog of umuD.

In some embodiments, the basic conjugative element is selected from a conjugative plasmid, an integrative conjugative element (ICE), and a mobile genetic element (MGE). In some embodiments, the basic conjugative element does not comprise a sequence encoding anti-defense genes or anti-defense-related genes in the leading region sequence, within about 35 kb from the oriT sequence and/or within the about 30 ORFs closest to the oriT. In some embodiments, the basic conjugative element does not comprise a sequence encoding anti-defense genes. In some embodiments, the basic conjugative element further comprises a sequence encoding a relaxase gene.

In some embodiments, the anti-defense nucleotide sequence further comprises a sequence including at least one single stranded DNA promoter, capable of initiating RNA transcription from single strand DNA. In some embodiments, the single stranded DNA promoter is selected from Frpo, ssiD, ssiE, ssi2, and ssi3.

In some embodiments, following insertion of the anti-defense nucleotide sequence, the conjugation efficiency of the improved conjugative element is increased by at least 10%, 15%, 20%, 25%, 30%, 25%, 40%, 45%, 50%, 60%, 70%, 80%, or 100% compared to the conjugation efficiency of the basic conjugative element.

In some embodiments, the basic conjugative element further comprises a cargo gene for transfer by conjugation, or the method further comprises inserting into the basic conjugative element or into the improved conjugative element a cargo gene for transfer by conjugation. In some embodiments, the cargo gene is selected from a gene capable of inhibiting or enhancing growth of a bacterial population, a gene capable of tagging bacteria, a gene capable of eradicating a bacterial population, a gene capable of causing bacteria to produce or sequester certain substances, a gene capable of eradicating antibiotics-resistant bacteria, a gene encoding an enzyme capable of degrading plant biomass, a gene which is a part of a pathway relevant to bacterial production of biofuel, a gene encoding an enzyme contributing to bioremediation, and a gene encoding an enzyme that can be used to sequester greenhouse gases.

In some embodiments, the method further comprises a step of transferring the improved conjugative element into donor bacterial cells. In some embodiments, the donor bacterial cells are Escherichia coli cells.

In some embodiments, the method further comprises a step of contacting recipient bacteria with the donor bacterial cells.

In some embodiments, the method is an in vitro method.

In some embodiments, the present invention provides a donor bacterial cell prepared by the method disclosed herein or comprising the improved conjugative element disclosed herein.

In some embodiments, the recipient bacteria are in a natural environment selected from an animal body, organ, or tissue; a plant; soil; marine environment; fresh water; and groundwater. In some embodiments, the animal is a human. In some embodiments, the recipient bacteria are in a human-designed environment selected from a bio-reactor, a water treatment plant, a water system, a hospital surface, a medical equipment, a vessel, a filter, and a pipe. In some embodiments, the human organ or tissue is selected from the respiratory system, the gastrointestinal tract, the urogenital system, the skin, and any organ including a microbiome.

In some embodiments, the recipient bacteria express a defense gene product which is inhibited by a product of the at least one anti-defense gene. In some embodiments, the defense gene is a gene of the CRISPR-Cas system and the anti-defense gene is an anti-CRISPR gene.

In some embodiments, the present invention provides an isolated nucleic acid molecule comprising an artificial anti-defense sequence of between about 20 kb and 35 kb in length, comprising:
more than one sequence encoding more than one anti-defense gene;
optionally at least one sequence encoding at least one anti-defense-related gene; and
at least one single-stranded DNA promoter driving the expression of the more than one anti-defense gene,
wherein
   the nucleic acid molecule does not comprise an oriT sequence;
   the anti-defense genes and anti-defense related genes, if present, are encoded on the same strand; and
   the nucleic acid molecule is optionally flanked on one side by at least one umu gene or homolog thereof.

In some embodiments, the present invention provides the isolated nucleic acid molecule disclosed herein for use in a method for increasing conjugation efficiency of a basic conjugative element which comprises an origin of transfer (oriT) sequence defining a leading region sequence, a T-strand, and a T-strand complement, the method comprising inserting the isolated nucleic acid molecule into the leading region sequence, thereby generating an improved conjugative element, wherein: the isolated nucleic acid molecule is inserted such that the anti-defense genes are inserted within about 35 kb from the oriT and/or within the about 30 open reading frames (ORFs) closest to the oriT, and are encoded on the T-strand complement; and conjugation efficiency of the improved conjugative element is increased compared to conjugation efficiency of the basic conjugative element.

In some embodiments, the present invention provides an improved conjugative element for use in a method of modulating recipient bacteria, wherein the improved conjugative element comprises:
an origin of transfer (oriT) sequence defining a leading region sequence, a T-strand, and a T-strand complement;
at least one anti-defense gene encoded on the T-strand complement, within the leading region sequence, within about 35 kb from the oriT and/or within the about 30 open reading frames (ORFs) closest to the oriT; and
a cargo gene capable of modulating the recipient bacteria,
and the recipient bacteria express a defense gene product which is inhibited by a product of the at least one anti-defense gene.

In some embodiments, the present invention provides a method for modulating recipient bacterial, the method comprising contacting the recipient bacteria with donor bacteria comprising a conjugative element, which comprises:
an origin of transfer (oriT) sequence defining a leading region sequence, a T-strand, and a T-strand complement;
at least one anti-defense gene encoded on the T-strand complement, within the leading region sequence, within about 35 kb from the oriT and/or within the about 30 open reading frames (ORFs) closest to the oriT; and
a cargo gene capable of modulating the recipient bacteria,
wherein the recipient bacteria express a defense gene product which is inhibited by a product of the at least one anti-defense gene.

In some embodiments, the recipient bacteria are in a natural environment. In some embodiments, the natural environment is an animal body, organ, or tissue. In some embodiments, the natural environment is a water body or water system.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures.

FIGS. 1A-1B. show enrichment of anti-defense genes in plasmid leading regions. FIG. 1A. Analysis of anti-defense proteins encoded on annotated plasmids. The x-axis is numbered by ORFs, starting from the origin of transfer (oriT), such that the 1 indicates the first ORF in the leading region and −1 indicates the first ORF in the lagging region. The y-axis denotes the average % of anti-defense genes out of the ORFs, combining well-characterized SOS inhibitor, anti-restriction, and anti-CRISPR genes, in a window including 5 ORFs upstream and 5 ORFs downstream. FIG. 1B. Breakdown of the anti-defense gene frequency according to their categories: SOS inhibitors, anti-restrictions, and anti-CRISPRs.

FIG. 2 shows anti-defense islands. Four representative anti-defense islands in leading regions of conjugative elements. The oriT location, where the conjugation transfer starts, are marked in red on the left. Genes are colored-coded according to their functional category: red: anti-defense, orange: anti-defense-related, blue: mobility (transfer genes), teal: gene without known association to anti-defense, grey: uncharacterized genes that were enriched in the leading regions, and white: uncharacterized genes. Frpo-type promoters are indicated by an arrow. Promoters with significant similarity to known Frpo sequences are marked with a solid arrow, Frpo candidates that were identified are marked with a dashed arrow, or dashed arrow with a star (*) for lower certainty candidates.

FIG. 3. Shows a suggested model of protection provided to the plasmids of the invention by the diverse anti-defense functions encoded on the leading region. Owing to ssDNA promoters, the anti-defense genes can be expressed at the very early stages of transfer to a recipient cell. During this phase, the bacterial immune response recruits its defense systems to prevent the entry of the transferred foreign DNA. Anti-CRISPRs encoded on the plasmid can inhibit CRISPR-Cas systems; SOS-inhibitors, such as PsiB protein, can repress the cell SOS-response by preventing the activation of RecA thus inhibiting the cleavage of LexA, an SOS response transcriptional repressor; Single-stranded binding (SSB) protein are involved in the SOS response inhibition mechanism, and may protect the transferred ssDNA from host nucleases. Methyltransferases (MTase), methylating the ssDNA can prevent recognition by the host restriction-modification (R-M) systems; Anti-restriction proteins can prevent DNA cleavage by R-M systems; and Antitoxins can neutralize host TA systems.

FIG. 4 shows conjugation frequencies for various configurations of an anti-defense gene and an ssDNA promoter. Conjugation frequency of an F plasmid carrying the anti-defense gene into a recipient containing a targeting SpyCas9 is depicted with grey bars while conjugation into a recipient with a non-targeting SpyCas9 is represented by white bars. The Y-axis represents the conjugation frequency (calculated as described in the Methods) on a log₁₀-scale, multiplied by 1,000,000. Error bars indicate standard errors from three biological replicates. The strains are as follows (from left): (1) Frpo No AcrIIA4, representing an F plasmid with the natural Frpo promoter knocked out and without an anti-CRISPR gene. (2) Lead: Frpo-AcrIIA4, consisting of Frpo and Anti-CRISPR AcrIIA4 in the leading region encoded on the T-strand complement; (3) Lagg: Frpo-AcrIIA4, with Frpo and AcrIIA4 in the lagging region encoded on the T-strand complement; and (4) Lead: AcrIIA4-Frpo rev, with AcrIIA4 and Frpo encoded on the T-strand (in reverse to the “usual” orientation of anti-defense genes).

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present invention is directed to improving conjugation efficiency of conjugative elements such as plasmids, based on the discovery by the inventors that anti-defense genes are concentrated in island-like clusters in the leading region of natural conjugative elements, and encoded on the strand complementary to the T-strand (T-strand being the strand that is nicked and transferred to recipient cell through conjugation) that results in their early expression during conjugation.

An intrinsic part of the arms race between conjugative elements and their host is the interplay between bacterial defense systems and the anti-defense systems encoded, e.g., on mobile genetic elements (MGEs). The results presented below provide a better understanding of strategies conjugative elements have developed during evolution to cope with the selective pressure of bacterial defense systems in a way that allows MGEs to rapidly overcome immunity and expand their distribution in the bacterial community.

The inventors show herein that an extensive repertoire of anti-defense genes, including anti-clustered regularly interspaced short palindromic repeats (CRISPR), anti-SOS, and anti-restriction genes, is encoded in the leading region of conjugative elements, which is the first region to be transferred to the recipient cell. Further, the anti-defense genes tend to cluster into “islands,” and reside downstream to promoters in an orientation that indicates that they could be expressed early while still in single-stranded DNA form. These results suggest that anti-defense islands on plasmids enable rapid protection against bacterial immunity and therefore successful establishment in the host.

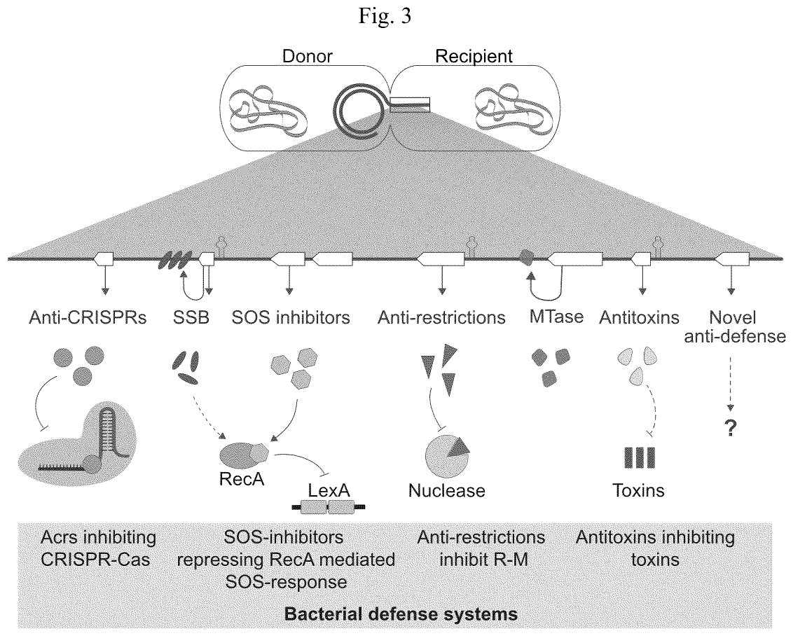

As can be seen from the examples, the inventors found that the leading regions of plasmids and other conjugative elements are highly enriched with anti-defense genes. Examination of these regions across an extensive genomic and metagenomic dataset revealed that various anti-defense systems in the leading region cluster together in “anti-defense islands”. These islands contain anti-CRISPRs, anti-restriction proteins, and SOS-inhibitor proteins, alongside anti-defense-related proteins, such as “orphan” methyltransferases, antitoxins, and SSB proteins. Uncovering anti-defense systems encoded in the leading regions suggests that these systems might be expressed very early upon entry to the recipient cell. This is supported by the presence of Frpo-type promoters, which allow expression from ssDNA. The combination of the location of the anti-defense genes and ssDNA promoters indicates they could be early expressed, well before the transfer is complete. This may enable rapid protection against the host defense systems during the initial establishment of the basic conjugative element in the host cell (FIG. 3).

Numerous gene families enriched in the anti-defense islands were uncharacterized. However, their location in this region strongly indicates that they likely have anti-defense-related functions. This could facilitate the discovery of new anti-defense systems, such as anti-CRISPR proteins, which are challenging to detect due to their small size and high variability. Focusing the search for these proteins in specific genomic loci, such as the leading region of conjugative elements, can contribute considerably to the efficient detection of anti-CRISPRs.

Intriguingly, a considerable fraction of the anti-defense islands is flanked by umu gene homologs, which encode translesion DNA synthesis polymerases. These genes are widespread on conjugative elements, and are also found in other MGEs, including the conjugative transposon Tn5252, phages, and prophages. Interestingly, they have been suggested to be involved in protecting plasmid and phage genomes by allowing DNA synthesis across otherwise unrepairable lesions. In the analysis, the umu gene homologs were almost exclusively encoded by the strand complementary to the anti-defense genes (i.e., on the T-strand), and are thus not expected to be expressed early with the anti-defense genes, since the anti-sense DNA strand must be produced before it can be transcribed into mRNA. Still, their abundance on various MGEs and their specific position at the edge of anti-defense islands in conjugative elements suggest they have a yet undetermined function in conjugation.

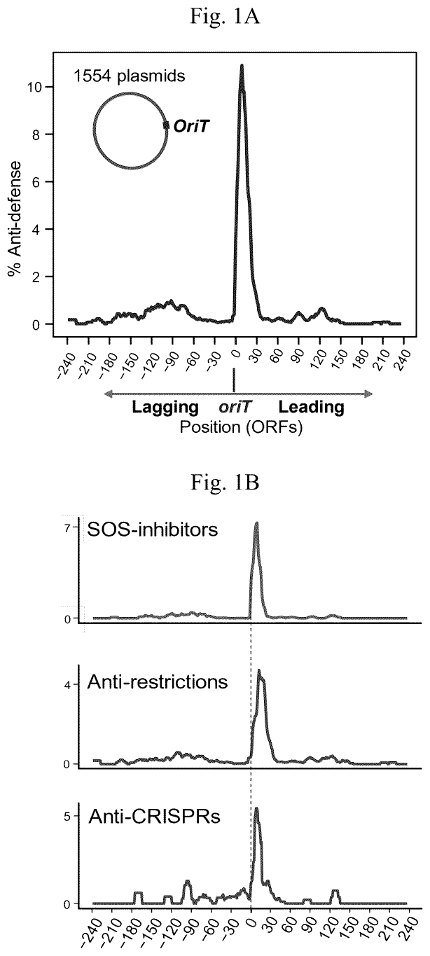

In addition to the highly significant enrichment of anti-defense genes in the leading region, in the 30 or so open reading frames (ORFs) closest to the oriT, a mild (statistically insignificant) enrichment of anti-defense genes was observed in the lagging region of conjugative elements (around −60 to −120 ORFs positions; FIG. 1). This may be due to the misidentification of the leading region based on the location of the oriT and the relaxase. Some sequences have several relaxosome genes and several sequences bearing similarity to known oriTs. In these cases, the leading region might be incorrectly identified.

The direction of transfer was determined by locating the oriT sequence and the relaxosome proteins (relaxase/TraM). Conjugative elements with unknown oriT sequences most likely hampered the ability to comprehensively retrieve conjugative elements. However, the robustness of the anti-defense gene location demonstrated in this study could be used to develop new approaches to identify oriT sequences based on the location of the relaxase or traM genes and the anti-defense islands. Such an approach, in combination with experimental testing, could significantly expand the set of known oriT sequences, providing a better characterization of conjugative elements.

A large set of plasmids was discarded from the analysis because a relaxase gene could not be identified in their sequence, and therefore the directionality of their transfer could not be defined. Over the last decade, several studies have shown that small plasmids that do not encode a relaxase can undergo transfer by utilizing relaxase-in trans. In these cases, a relaxase from another plasmid in the same cell can mobilize both plasmids. The findings can also be used to define the leading region and the direction of transfer of plasmids lacking a relaxase gene by detecting anti-defense genes, Frpo promoters, and an oriT in their sequence. This allows a better understanding of early expressed genes in small plasmids encoding only an oriT, which is highly important as they have been shown to carry numerous antimicrobial resistance genes (ARGs) and make up the majority of plasmids. Interestingly, several early expressed genes in phages have also been associated with anti-defense functions.

Accordingly, in some embodiments, the present invention provides a method for increasing conjugation efficiency of a basic conjugative element which comprises an origin of transfer (oriT) sequence defining a leading region sequence, a T-strand, and a T-strand complement (a strand complementary to the T-strand), the method comprising inserting into the leading region sequence an anti-defense nucleotide sequence comprising a sequence encoding at least one anti-defense gene, thereby generating an improved conjugative element, wherein: the at least one anti-defense gene is inserted such that it is encoded on the T-strand complement; and the conjugation efficiency of the improved conjugative element is increased compared to the conjugation efficiency of the basic conjugative element.

Many conjugative elements in use today for biotechnological applications do not comprise anti-defense genes, or their anti-defense genes are not ideally positioned, making them susceptible to the host defense systems, thereby causing less than optimal rates of conjugation. Adding sequences encoding anti-defense genes in the correct position and at the correct orientation for early expression, according to the present invention, would lead to a higher conjugation efficiency for such conjugative elements. Examples for such candidate conjugative elements include RP4 plasmid and derivative thereof, pTA-Mob (Soltysiak et al., Int. J. Mol. Sci. 2019, 20, 5212) and derivative thereof, and F-plasmid (such as the F derivative pOX38, accession NZ_MF370216.1).

The term “basic conjugative element” as used herein relates to any mobile genetic element capable of conjugation. This definition includes both conjugative plasmids and integrative conjugative elements (ICEs) that encode the entire transport machinery, as well as mobile genetic elements (MGEs) that contain only the relaxase gene and an oriT sequence but lack the machinery required for self-transfer. MGEs, also termed “mobilizable plasmids”, can be transferred using the conjugation machinery of a co-residing conjugative element. It should be clarified that the basic conjugative element related to herein is the basic conjugative element prior to modification according to the invention. Following modification, the conjugative elements is referred to as an improved conjugative element, or an artificial improved conjugative element.

Accordingly, in some embodiments, the basic conjugative element is any conjugative plasmid. In some embodiments, the basic conjugative element is selected from an RP4 plasmid, a pTA-Mob, an F-plasmid, an RK2-based conjugative plasmid, an IncI plasmid (such as a TP114 plasmid), and derivatives thereof. In some embodiments, the derivative thereof does not include anti-defense gene in addition to the anti-defense genes included in the original plasmid.

In some embodiments, the basic conjugative element is a native, or natural element.

In some embodiments, the basic conjugative element is a synthetic, or recombinant, element.

In some embodiments, the basic conjugative element has a length of at least about 2, 3, 4, 5, 10, 50, or 100 kb.

In some embodiments, the basic conjugative element is selected from a conjugative plasmid, an integrative conjugative element (ICE), and a mobile genetic element (MGE).

A relaxase gene is often encoded by a sequence in the lagging region of conjugative elements, close to the oriT. The relaxase gene is part of the relaxosome complex that facilitates plasmid transfer during bacterial conjugation. The relaxase is responsible for beginning the conjugation process by cutting at the nic site via transesterification. This nicking results in a DNA-Protein complex with the relaxosome bound to a single strand of the plasmid DNA and an exposed 3′ hydroxyl group. Relaxase also unwinds the plasmid being conjugated with its helicase properties.

Accordingly, in some embodiments, the basic conjugative element further comprises a sequence encoding a relaxase gene. In some embodiments, the method further comprises inserting a sequence encoding a relaxase gene to the lagging strand of the basic conjugative element.

Additionally, according to the present invention, in cases where the location of the oriT and/or the direction of transfer are difficult to determine, the position of the sequence encoding the relaxase gene, together with the position of an anti-defense island may be used in order to determine the position of the oriT and the direction of transfer.

The term “improved conjugative element”, as used herein, relates to a basic conjugative element which has been improved by adding at least one anti-defense gene as described in the invention.

The term “leading region” refers to a region encompassing a sequence of about 20-35 kb starting from the oriT, which is transferred first to the recipient bacteria. In some embodiments, the leading region encompasses about 15-35 kb, 15-30 kb, 15-25 kb, 20-35 kb, or 20-30 kb, starting from the oriT.

The term “lagging region” refers, in a parallel way to the leading region, to the region transferred last to the recipient.

The leading and the lagging regions are located on opposite sides of the oriT.

The origin of transfer (oriT) is a short sequence ranging from 10-500 base pairs in length, which is necessary for the transfer of DNA from a bacterial donor to a bacterial recipient during bacterial conjugation.

It is noted that for determination of leading and lagging regions, as well as the T-strand and T-strand complement, the direction of transfer needs to be determined. A non-limiting example for determining the direction of transfer is by determined by locating the oriT sequence and the relaxosome proteins (relaxase/TraM). Relaxosome components are typically encoded in the lagging region adjacent to oriT. Thus, the leading region is defined as the region adjacent to the oriT on the other side, i.e., the side opposite to the side encoding the relaxosome components.

The term “T-strand” refers to the strand that is nicked and transferred through conjugation to the recipient cell. For genes encoded on the T-strand, the T-strand is the coding strand for these genes.

The term “T-strand complement” refers to the strand complementary to the T-strand. For genes encoded on the T-strand complement, the T-strand complement is the coding strand for these genes. Upon conjugation, mRNA for these genes is readily transcribed from the T-strand (which is the anti-sense strand), and therefore such genes may be expressed early during transfer, and even before conjugation is complete. It was found in the present invention that anti-defense genes are usually located in the leading region and encoded on the T-strand complement. Their early expression allows them to counter the cellular defense system and protect the transferred plasmid.

The term “coding strand” relates to the strand which comprises codons, and which has the same sequence as the mRNA (mutatis mutandis).

The term “anti-defense gene”, as used herein, encompasses genes which counter the action of bacterial defense systems, as well as genes which are prevalent in leading regions of conjugative elements.

The bacterial defense systems countered by anti-defense genes suitable for the present invention include, but are not limited to, CRISPR-Cas, R-M (restriction-modification) systems, and the SOS system. Such anti-defense genes include anti-CRISPR (acr) genes such as acrIIA4, acrIC6, acrIF16, acrIE9, acrIB, and acrIIA21; anti-restriction genes such as ocr, ardA, ardB, klcAHS, and darB; and SOS inhibitor genes such as psiA and psiB.

In some embodiments, the anti-defense gene is a gene known to counter the action of bacterial defense systems.

In some embodiments, the at least one anti-defense gene is selected from AcrIE1, AcrIE2, AcrIE3, AcrIE4, AcrIE4-F7, AcrIE5, AcrIE6, AcrIE7, AcrIE8, AcrIE9, AcrIF1, AcrIF2, AcrIF3, AcrIF4, AcrIF5, AcrIF6, AcrIF7, AcrIF8, AcrIF9, AcrIF10, AcrIF11, AcrIF12, AcrIF13, AcrIF14, AcrIF15, AcrIF16, AcrIF17, AcrIF18, AcrIF19, AcrIF20, AcrIF21, AcrIF22, AcrIF23, AcrIF24, AcrIC1, AcrIF2/C2, AcrIC3, AcrIC4, AcrIC5, AcrIC6, AcrIC7, AcrIC8, AcrIC9, AcrIC10, AcrIC11, AcrIIA1, AcrIIA2, AcrIIA3, AcrIIA4, AcrIIA5, AcrIIA6, AcrIIA7, AcrIIA8, AcrIIA9, AcrIIA10, AcrIIA11, AcrIIA12, AcrIIA13, AcrIIA14, AcrIIA15, AcrIIA16, AcrIIA17, AcrIIA18, AcrIIA19, AcrIIA20, AcrIIA21, AcrIIA22, AcrIIA23, AcrIIA24, AcrIIA25, AcrIIA26, AcrIIA27, AcrIIA28, AcrIIA29, AcrIIA30, AcrIIA31, AcrIIA32, AcrIII-1, AcrIIIB1, AcrIIC1, AcrIIC2, AcrIIC3, AcrIIC4, AcrIIC5, AcrIIC6, AcrID1, AcrVA1, AcrVA2, AcrVA3, AcrVA4, AcrVA5, AcrVIA1(Lse)_num1, AcrVIA2, AcrVIA3, AcrVIA4, AcrVIA5, AcrVIA6, AcrVIA7, AcrVIA1 (Lse)_num2, AcrIB, AcrVIB1, aca1, aca2, aca3, aca4, aca5, aca6, aca7, aca8, aca9, aca10, aca11, aca12, aca13, vcrx091, vcrx092, vcrx093, ardA, ardB, ardC, ArsR, ardU, Ugi, KlcAHS, darA, darB, DdrA, DdrB, Hdf, Ulx, Ocr, ardD, “merR,”, vcrx089, vcrx090, psiA, psiB, Acb1, Acb2, Tad1, Tad2, Apyc1, and Had1.

In some embodiments, the at least one anti-defense gene is an anti-CRISPR gene; an anti-restriction gene, and/or an SOS inhibitor gene. In some embodiments, the anti-CRISPR (acr) gene is selected from acrIIA4, acrIC6, acrIF16, acrIE9, acrIB, and acrIIA21. In some embodiments, the anti-restriction gene is selected from ocr, ardA, ardB, klcAHS, and darB. In some embodiments, the SOS inhibitor is selected from psiA and psiB.

In some embodiments, the at least one anti-defense gene has at least about 80%, 85%, 90%, 95%, or 99% nucleic acid sequence identity with any of the genes listed above. Based on results of the experiments provided herein (see Examples 1-2) which analyzed many plasmid sequences from databases, a list of genes is provided below, which may be suitable for use with the invention, as anti-defense genes. For some of these genes, anti-defense activity has been predicted by an in silico analysis, see Table 2. Genes included in the below lists were those for which the enrichment in the leading region of plasmids was statistically significant and that were encoded on the relevant strand.

In some embodiments, the anti-defense gene encodes a protein having a UniProt accession selected from Q89Z22, A0A2I0FQS0, A0A0K2CSG1, E9LLV6, A0A7W5VW31, A0A4Y1VNF6, A0A395L6C2, A0A3P5HF59, A0A0A8V8B5, A0A402TQZ8, A0A3J8VOU3, A0A6B7PZD7, BOBL17, E9LMF8, A0A0D8L5B1, A0A629I8E3, A0A377EB90, K6BUI3, A0A1L4J4T2, A0A934WQS3, A0A3D3BKI5, A0A218SV17, A0A6H1Q5R4, A0A2N4Z006, A0A4S4ZCT9, A0A6S4YC94, A0A6C0L1U9, A0A0K4WQ54, A0A066Q7F0, U9YKL4, M1EXT5, A0A3P5DT87, A0A7U9ARL0, A0A3D1PQA7, U6RG21, A0A060VLU1, A0A2P9AY37, A0A2A6P1X5, Q08JH9, A0A220SXH4, C3S7V6, A0A927HTW9, A0A3E4UK73, A0A0H2XJE0, A0A7S8ELB6, A0A0N1C5J9, A0A3Q8YRH2, A0A0U1QT64, A0A0U1QTC2, A0A377BE37, A0A660E2S4, A0A7T4LT33, A0A0T9N008, A0A0T9N0K3, A0A3Y9C7Q4, A0A1ZINXV3, B5SU32, A0A1CIEM56, S5NNU9, K6B4H9, A0A1G6A012, E9TMD2, Q02885, A0A3R1BGK4, A0A807NGC1, A0A2S4Q769, A0A0T6ZGI4, A0A209AHL8, N8QA52, A0A0J9FC28, E0J397, A0A403MPA0, A0A1Q3QK10, A0A443XDG2, A0A2D3TAG3, A0A316N715, A0A9J9H852, A0A2A6HSK2, R9H067, A0A075MFN2, A0A075MFN2, AOA7R7HET7, A0A075MFN2, A6X7NO, A0A944AX61, A0A0T9QCQ4, A0A0T9RM70, W0EW90, Q8DX38, Q9JMS6, A0A2W5QXB4, F3B3P7, A0A6L6YBB5, A0A4Y7QQA5, A0A395YM67, A0A0H3MFY0, A0A2N4Z900, A0A750MRT0, A0A1X9WZZ3, E7BTG5, U9XRN8, A0A015X6K6, A0A4Y1WXJ5, A0A415DE31, H1ZXV2, A0A0P7SU86, R6U4Y9, Q3ZU45, A0A2V3VPD9, A0A413C521, A0A9P2WH83, A0A2L1UZG8, A0A2L1UZ22, A0A1H1QVI4, A0A4R1YJE5, Q9L5L3, I2J4S5, A0A1W6AS68, A0A1E7Z4T1, D4E9W8, A0A3G4RJ75, A0A0K2CS29, A0A899NJG9, G9G282, A0A522DZX4, A0A2R4PEL7, A0A3R0XF69, A0A1L1PWL3, A0A6N2XUU1, A0A1E7Z3N7, C3KQZ1, A0A5U3IUX1, N9QD66, C1J8J6, A0A6M3HDA8, A0A1E7Z341, H3RM30, A0A0A3TKL8, A0A4R3RGK8, A0A377BFD5, F0QZH4, A0A5C5CX89, A0A6Y2KPU3, A0A7U5G6W8, A0A1N6VQ23, A0A3N4N999, A0A793HHF9, A0A1C3WDH0, A0A731NS67, A0A2I8NZ96, A0A3Z1VAZ4, A0A2L2WMI3, A0A614DZN8, A0A701ZQX0, A0A1C5U060, A0A2T7VXY6, KOZ6R5, A0A941T9P3, A0A2V1H6L5, A0A318P318, G7YYQ0, A0A482LZH2, AOA7Y8CQW0, A6N5S2, A0A6L3K1B2, A0A5H7NWA4, A0A8D5CPH1, A0A514F012, A0A7W3KUZ1, L0R319, A0A486BEG4, A0A533HNH6, A0A0A3YNE5, A0A246JSA1, A0A923EQJ0, A0A3R0JMS4, A0A6W0P441, D2J5V5, A0A6G1WUF3, A0A7X6FL32, A0A1I4VS10, Q71TR0, and A0A645CD74.

In some embodiments, the anti-defense gene encodes a protein having at least about 80%, 85%, 90%, 95%, or 99% amino acid sequence identity with any of the protein sequences listed above.

In some embodiments, the anti-defense nucleotide sequence further comprises a sequence encoding at least one anti-defense-related gene, and the at least one anti-defense-related gene is encoded on the same strand as the anti-defense genes, namely on the T-strand complement.

The term “anti-defense-related gene” as used herein relates either to genes not directly opposing the bacterial defense system but rather supporting the action of anti-defense genes, or to genes that have additional roles in the cell or have different functions under different conditions. Such genes include methyltransferases, single-strand DNA-binding proteins (SSBs), toxins and/or antitoxins, such as abiEii-abiEi and higB-higA TA systems, and hipB antitoxin.

In some embodiments, the at least one anti-defense-related gene is a methyltransferase, a single-strand DNA-binding protein (SSB), a toxin, and/or an antitoxin, a DNA repair gene.

In some embodiments, the at least one anti-defense-related gene is selected from hokA, hokB, hokC_D, hokE, sokB, sokC, symE, symR, IdrA_B_C_D, rdlA_B_C_D, tisB, istR, ibsA, ibsB, ibsC, ibsD, ibsE, sibA, sibB, sibC, sibD, sibE, shoB, ohsC, ralR, ralA, mazF, mazE, ndoAI, chpB, chpS, yhaV, prlF, relE, relB, stbD, yafQ, dinJ, yoeB, yefM, hipA, hipB, hicA, hicB, yafO, yafN, hha, tomB, pspC, pspB, ccdB, ccdA, parE1_3_4, parE2, parD1_3_4, parD2, doc, phd, higB, higB-1, higA, higA-1, mqsR, mqsA, mvpA, mvpT, vapC, vapB, pezT, pezA, yobL, yobK, yokI, yokJ, yqcG, yqcF, yxiD, yxxD, fitB, fitA, BC_0920, BC_0921, tabA, mcbA, bssS, bssR, bhsA, ftsZ, mreB, parC, parE, EARS, nob1, AbiQ, cbtA, cheA, cptA, cptB, ghoT, ghoS, hsdR, hsdS, hsdM, E3.1.21.4, dam, ccrM, K07317, K07318, yhdJ, E2.1.1.72, DNMT1, E2.1.1.113, res, mod, mcrA, mcrB, mcrC, and mrr.

In some embodiments, the anti-defense-related gene has at least about 80%, 85%, 90%, 95%, or 99% nucleic acid sequence identity with any of the genes listed above.

In some embodiments, the anti-defense gene and/or anti-defense-related gene are prevalent in leading regions of conjugative elements. In some embodiments, the anti-defense gene and/or anti-defense-related gene are prevalent within about 35 kb or within about 30 ORFs from the oriT of conjugative elements. In some embodiments, the anti-defense gene and/or anti-defense-related gene are at least about 5, 10, 15, or 20 times more prevalent in leading regions of conjugative elements, namely within about 35 kb from the oriT, or within about 30 ORFs from the oriT, than in lagging regions of conjugative elements. In some embodiments, the anti-defense gene and/or anti-defense-related gene are about 5-25, 5-20, 5-15, or 5-10 times more prevalent in leading regions of conjugative elements, namely within about 35 kb from the oriT, or within about 30 ORFs from the oriT, than in lagging regions of conjugative elements.

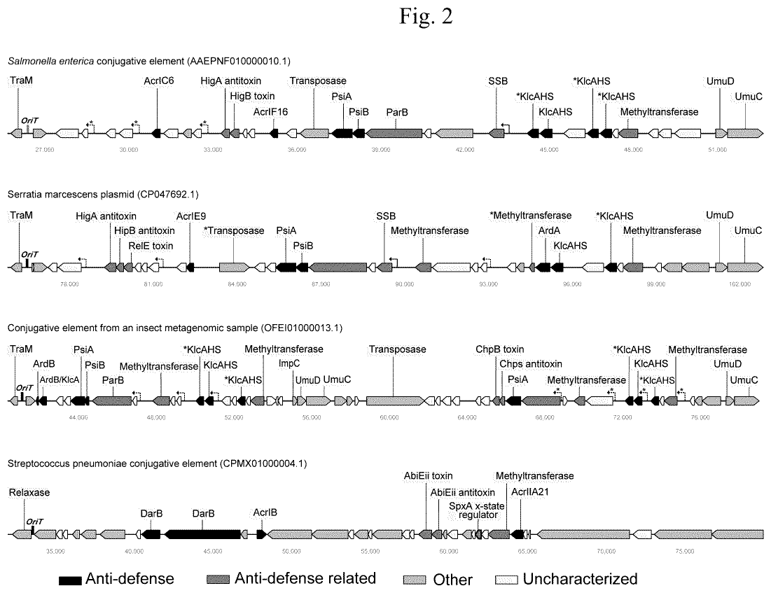

Anti-defense genes and anti-defense-related genes are often organized in island-like clusters located close to the oriT, such as within approximately 20-35 kb, in the leading region. Such clusters may be herein referred to as “anti-defense islands”. Anti-defense islands are sometimes flanked, in the oriT-distal region, by translesion DNA synthesis polymerases (such as DNA polymerase V), which are also known as umuC and umuD genes. Examples for conjugative elements containing such islands include Salmonella enterica conjugative element (GenBank accession: AAEPNF010000010.1), Serratia marcescens plasmid (GenBank accession: CP047692.1), insect metagenomic sample conjugative element (GenBank accession: OFEI01000013), and Streptococcus pneumoniae conjugative element (GenBank accession: CPMX01000004.1) (FIG. 2).

In some embodiments, the length of the anti-defense nucleotide sequence, which may be an anti-defense island, is about 10-35 kb. In some embodiments, the length of the anti-defense nucleotide sequence is about 15-35 kb, 20-35 kb, 25-35 kb, 15-30 kb, 15-27 kb, 15-25 kb, 20-28 kb, 23-27 kb, 25-27 kb.

In some embodiments, the anti-defense nucleotide sequence is inserted into the leading region, within about 35 kb, 30 kb, 25 kb, 20 kb, 15 kb, 10 kb, 5 kb, 1 kb, 100 bp, or 50 bp from the oriT.

In some embodiments, by insertion of the anti-defense nucleotide sequence, the at least one anti-defense gene and/or at least one anti-defense-related gene is inserted within about 35 kb, 30 kb, 25 kb, 20 kb, 15 kb, or 10 kb from the oriT, in the leading region.

As further found by the inventors (see FIG. 1), the anti-defense and anti-defense-related genes are concentrated within the about 30 ORFs closes to the oriT on the leading side.

Accordingly, in some embodiments, the anti-defense nucleotide sequence is inserted into the leading region sequence within the about 60, 50, 40, 30, 20, 10, or 5 ORFs closest to the oriT. In some embodiments, the anti-defense nucleotide sequence is inserted into the leading region sequence within the about 30 ORFs closest to the oriT.

In some embodiments, by insertion of the anti-defense nucleotide sequence, the at least one anti-defense gene and/or at least one anti-defense-related gene is inserted within the about 60, 50, 40, 30, 20, 10, or 5 ORFs closest to the oriT, in the leading region. In some embodiments, by insertion of the anti-defense nucleotide sequence, the at least one anti-defense gene and/or at least one anti-defense-related gene is inserted within the about 30 ORFs closest to the oriT, in the leading region.

In some embodiments, the at least one anti-defense gene is a single anti-defense gene. In some embodiments, the at least one anti-defense gene is at least two anti-defense genes. In some embodiments, anti-defense nucleotide sequence comprises at least one anti-defense gene and at least one anti-defense-related gene, i.e. the anti-defense nucleotide sequence comprises an anti-defense island.

In some embodiments, the at least one anti-defense gene is at least two, at least three, at least four, or at least five anti-defense genes. In some embodiments, the anti-defense nucleotide sequence comprises at least one anti-defense gene, and at least two, at least three, at least four, or at least five anti-defense-related genes.

In some embodiments, the at least one anti-defense gene is at least two, at least three, at least four, or at least fine anti-defense-related genes.

In some embodiments, when the anti-defense nucleotide sequence comprises two or more anti-defense genes or at least one anti-defense gene and at least one anti-defense-related gene, the anti-defense genes and anti-defense-related genes (if present) are encoded on the same strand, i.e. on the T-strand complement.

In some embodiments, when the anti-defense nucleotide sequence comprises an anti-defense gene and at least one additional gene selected from an anti-defense gene and an anti-defense-related gene, at least one of the additional genes is encoded on the same strand as the anti-defense gene, i.e. on the T-strand complement.

In some embodiments, the anti-defense nucleotide sequence comprises an anti-CRISPR gene and an anti-restriction gene. In some embodiments, the anti-defense nucleotide sequence comprises an anti-CRISPR gene, an anti-restriction gene, and an SOS inhibitor gene.

In some embodiments, the at least one anti-defense genes are acrIE9 and ardA.

In some embodiments, the at least one anti-defense genes are acrIE9 and ardB.

In some embodiments, the at least one anti-defense genes are acrIE9 and klcHS.

In some embodiments, the at least one anti-defense genes are acrIF11 and ardA.

In some embodiments, the at least one anti-defense genes are acrIF11 and ardB.

In some embodiments, the at least one anti-defense genes are acrIF11 and klcHS.

In some embodiments, the at least one anti-defense genes are acrIE9 and acrIF11.

In some embodiments, the at least one anti-defense gene is acrIE9 and the at least one anti-defense-related gene is a methyltransferase.

In some embodiments, the basic conjugative element does not comprise a sequence encoding anti-defense genes. In some embodiments, the basic conjugative element does not comprise a sequence encoding anti-defense-related genes.

In some embodiments, the basic conjugative element does not comprise a sequence encoding anti-defense genes within about 35 kb from the oriT sequence. In some embodiments, the basic conjugative element does not comprise a sequence encoding anti-defense genes within about 35 kb from the oriT sequence. In some embodiments, the basic conjugative element does not comprise a sequence encoding anti-defense genes or anti-defense-related genes within about 35 kb, 30 kb, 25 kb, 20 kb, 15 kb, or 10 kb from the oriT sequence. In some embodiments, the basic conjugative element does not comprise a sequence encoding anti-defense genes or anti-defense-related genes within the 30 ORFs or so closest to the oriT.

In it noted that the basic conjugative element may comprise anti-defense genes or anti-defense related genes. However, the conjugation efficiency may be improved by adding anti-defense genes or anti-defense-related genes according to the present invention, such as anti-defense genes or anti-defense genes mentioned herein, or that the anti-defense genes or anti-defense-related genes are added in a position and orientation relative to the oriT, as described herein.

In some embodiments, the anti-defense nucleotide sequence further comprises a sequence encoding at least one umu gene or a homolog thereof, which is positioned (after insertion) farther away from the oriT compared to the at least one anti-defense gene. In some embodiments, the at least one umu gene is at least two umu genes. In some embodiments, the sequence encoding the at least one umu gene or homolog thereof is farther away from the oriT compared to the at least one anti-defense-related gene. In some embodiments, the at least one umu gene is a homolog of umuC and/or a homolog of umuD. In some embodiments, the umu genes are encoded on the strand complementary to the strand encoding the anti-defense genes.

The term “single stranded DNA promoter” as used herein relates to a promoter capable of transcribing RNA from single stranded DNA. These promoters can adopt a stem-loop structure such that the regions of the −10 and −35 elements mimic a double-strand conformation, allowing their recognition by the host RNA polymerase. The sequences at −10 and −35 of promoters found within the anti-defense islands are similar to the consensus sequences TATAAT and TTGACA, respectively. Additionally, some putative promoters also harbor a sequence similar to the UP element upstream of the −35 element (AAAAATTTTTT). Examples of single stranded DNA promoters include Frpo, ssiD and ssiE in F plasmid, and ssi2 and ssi3 in the IncI1 plasmid Col1b-9. It was therefore proposed that Frpo may serve as a single-stranded promoter that allows early expression of the leading region genes.

In some embodiments, the anti-defense nucleotide sequence further comprises a sequence including at least one single stranded DNA promoter, capable of initiating RNA transcription from single strand DNA. the single stranded DNA promoter is selected from Frpo, ssiD, ssiE, ssi2, and ssi3.

In some embodiments, the single stranded DNA promoter drives transcription of at least one anti-defense gene. In some embodiments, the single stranded DNA promoter drives transcription of at least one anti-defense-related gene.

Conjugation efficiency may be determined by any suitable method, such as, but not limited to, by determining the amount of bacteria resistant to antibiotics, which is conferred by a marker transferred by the conjugation.

Accordingly, in some embodiments, following insertion of the anti-defense nucleotide sequence according to the invention, the conjugation efficiency of the improved conjugative element is increased by at least 10%, 15%, 20%, 25%, 30%, 25%, 40%, 45%, 50%, 60%, 70%, 80%, or 100% compared to the conjugation efficiency of the basic conjugative element, prior to insertion of the anti-defense nucleotide sequence. In some embodiments, following insertion of the anti-defense nucleotide sequence according to the invention, the conjugation efficiency of the improved conjugative element is increased to at least about 10, 20, 50, 10, 500, 1000, 2000, 3000, or 5000 fold compared to the conjugation efficiency of the basic conjugative element, prior to insertion of the anti-defense nucleotide sequence.

In some embodiments, the basic conjugative element further comprises a cargo gene for transfer by conjugation.

In some embodiments, the method further comprises inserting into the basic conjugative element or into the improved conjugative element a cargo gene for transfer by conjugation.

The term “cargo gene”, as used herein, relates to any gene that is not an anti-defense gene, and may be transferred into bacteria by conjugation. Non-limiting examples for useful cargo genes include genes capable of modulating characteristics of recipient bacteria, including, for example, genes capable of inhibiting or enhancing growth of certain bacterial populations, genes capable of tagging specific bacteria, genes capable of eradicating certain bacterial populations, or causing bacteria to produce or sequester certain substances, etc. More specific examples include plasmid carrying systems such as CRISPR-Cas targeting antimicrobial resistance genes; genes capable of eradicating resistant bacteria; genes encoding enzymes such as cellulases and hemicellulases that degrade plant biomass or are part of other pathways relevant to bacterial production of biofuel; genes encoding enzymes contributing to bioremediation, for example contaminants degradation using lipases, proteases, laccases, hydrolases, dehalogenases, and dehydrogenases; genes encoding enzymes that can be used to sequester greenhouse gases, such as rubisco or carbonic anhydrase for CO₂ sequestration, and methane monooxygenase for methane sequestration.

The improved conjugative elements of the invention may be especially useful for certain applications in which there is a need to modulate certain recipient bacteria which express defense genes, which may inhibit expression of transferred genes.

First, for using the improved conjugative elements of the invention in conjugation, they must be present in a donor bacteria. It is appreciated that the modification of the conjugative elements to obtain improved conjugative elements may be conducted in another bacterial strain or culture, and conjugated (or otherwise transferred) into the donor bacteria.

The term “donor bacteria”, as used herein, relates to bacteria carrying the improved conjugative element of the invention, and which are capable of transferring it to recipient bacteria by conjugation.

The term “recipient bacteria”, as used herein, relates to bacteria which the invention seeks to modulate, such as bacteria in a human-designed or a natural environment. These bacteria are capable of receiving the improved conjugative element of the invention from the donor bacteria by conjugation. The recipient bacteria typically include a defense system, and the improved conjugative element of the invention preferably includes at least one anti-defense gene which is capable of inhibiting or countering the action of the anti-defense system of the recipient bacteria.

Accordingly, in some embodiments, the method further includes a step of transferring the improved conjugative element into a donor bacterial cell.

The transferring may be conducted by any suitable method, such as conjugation or transformation.

The donor bacteria may be gram-negative or gram-positive bacteria, as well as Archaea. In some embodiments, the donor bacteria are selected from commonly-used bacteria such as Escherichia coli, Bacillus subtilis, Mycobacterium Tuberculosis, Streptomyces, and Salmonella serotypes; from gut bacteria, such as bacteria belonging to the genera Prevotella, Bacteroides, Faecalibacterium, Ruminococcus, Blautia, and Clostridium; and from major resistance bacteria, such as Staphylococcus aureus, Enterococcus, Pseudomonas aeruginosa, Klebsiella pneumonia, and Acinetobacter baumannii.

In some embodiments, the method is an in vitro method.

In some embodiments, the method further comprises a step of contacting recipient bacteria with the donor bacterial cells.

The term “contacting”, as used here, generally relates to adding donor bacteria in the environment in which recipient bacteria are present, so that the donor bacteria may transfer the improved conjugative element they carry, by conjugation, into the recipient bacteria. The environment may be any suitable environment containing (recipient) bacteria, such as a natural environment or a human-designed environment. Such environments include, but are not limited to, organs or tissues of a body, as well non-animal environments in which bacteria may be present, including surfaces, soil, air, and water.

Some of the above-mentioned applications involving modulating bacteria relate to bacteria in natural environments or in human-designed environments.

In some embodiments, the natural environment is a living, or organic, natural environment, such as animal body, organ, or tissue; or a plant or plant part. In some embodiments, the natural environment is a non-organic natural environment, such as soil, air, or water such as freshwater, a marine environment, a body of water, or groundwater.

In some embodiments, the human-designed environments include bio-reactors, water treatment plants, water systems, hospital surfaces, medical equipment, vessels (e.g. ships), filters (e.g., percolating filters and slow sand filters), pipes, and the like.

Non-limiting examples for an animal include a mammal, such as a human; a bird, or an insect.

Non-limiting examples for organs or tissues include respiratory system, gastrointestinal tract, urogenital system, skin, mucous, or any animal environment including a microbiome.

The recipient bacteria may be gram-negative or gram-positive bacteria, as well as Archaea.

In some embodiments, the recipient bacteria express a defense gene product which is inhibited by a product of the at least one anti-defense gene.

For example, in some embodiments, the defense gene is a gene encoding a CRISPR-Cas system product (e.g., Cas9), and the anti-defense gene is an anti-CRISPR gene (e.g., AcrIIA4, AcrIIA16).

Another example is when the defense gene is an anti-restriction gene (e.g., ardB) and the defense gene is a restriction-modification (R-M) system gene, such as a type I R-M EcoKI enzyme present in MG1655.

Non-limiting examples for recipient bacterial species carrying specific defense systems include bacteria carrying CRISPR-Cas systems, including E. coli, P. aeruginosa, S. thermophilus, S. mutants, S. pyogenes, P. furiosus, S. epidermidis, and M. tuberculosis; bacteria carrying restriction-modification systems, including E. coli and N. meningitidis; bacteria carrying SOS-response systems, including E. coli; and bacteria carrying DISARM systems, including Burkholderia pseudomallei and Bacillus paralicheniformis. Accordingly, conjugative elements carrying appropriate anti-defense genes, as disclosed herein, may be used for each bacterial species, as needed.

It is noted that although different bacteria harbor different defense systems, according to the present invention, the rate of conjugation also benefits from adding general anti-defense genes or anti-defense-related genes that do not counter a specific defense system, such as methyltransferases, single-strand DNA-binding proteins (SSB), which can prevent degradation by different types of nucleases, and SOS-inhibitors (psiA and psiB) which can inhibit the SOS-response common in various bacteria.

Accordingly, in some embodiments, the identity of the anti-defense genes(s) and/or anti-defense-related gene(s) does not depend on the identity of the recipient bacteria.

In some embodiments, the identity of the anti-defense genes(s) and/or anti-defense-related gene(s) depends on the identity of the recipient bacteria.

In some embodiments, the identity of the anti-defense gene or anti-defense-related gene depends on the defense systems present in the recipient bacteria into which conjugation of the basic conjugative element is desired. In some embodiments, the anti-defense gene or anti-defense-related gene functions to counter defense systems present in the recipient bacteria. For example, an anti-restriction gene (e.g., ardB) could be added to a basic conjugative element when the recipient bacterial strain has a restriction-modification (R-M) system, such as in MG1655, which harbors a type I R-M EcoKI enzyme. Another example is adding an anti-CRISPR gene when the recipient has a CRISPR/Cas system.

In some embodiments, the present invention provides donor bacterial cells comprising the improved conjugative elements disclosed herein.

These donor bacterial cells may be generated by the methods disclosed herein.

Definitions and embodiments mentioned above and which may be relevant to the bacteria also apply here, and vice versa. Some particularly relevant embodiments may be pointed out or explicitly repeated.

In some embodiments, the present invention provides the donor bacterial cells disclosed herein for use in method of modulating recipient bacteria as disclosed hereinbelow, wherein the recipient bacteria express a defense gene product which is inhibited by a product of the at least one anti-defense gene.

In some embodiments, the present invention provides the donor bacterial cells disclosed herein for use in method of treating a disease or disorder associated with recipient bacteria as disclosed hereinbelow, wherein the recipient bacteria express a defense gene product which is inhibited by a product of the at least one anti-defense gene.

Definitions and embodiments mentioned herein and which may be relevant to the use of the bacteria also apply here, and vice versa. Some particularly relevant embodiments may be pointed out or explicitly repeated.

In some embodiments, the present invention provides an isolated nucleic acid molecule comprising an artificial anti-defense sequence of between about 20 kb and 35 kb in length, comprising:
more than one sequence encoding more than one anti-defense gene;
optionally at least one sequence encoding at least one anti-defense-related gene; and
at least one single-stranded DNA promoter driving the expression of the more than one anti-defense gene,
wherein
   the nucleic acid molecule does not comprise an oriT sequence;
   the anti-defense genes and anti-defense related genes, if present, are encoded on the same strand; and
   the nucleic acid molecule is optionally flanked on one side by at least one umu gene or homolog thereof.

Definitions and embodiments mentioned above and which may be relevant to the isolated nucleic acid molecule also apply here, and vice versa. Some particularly relevant embodiments may be pointed out or explicitly repeated.

In some embodiments, the present invention provides the isolated nucleic acid molecule disclosed herein, for use in a method for increasing conjugation efficiency of a basic conjugative element which comprises an origin of transfer (oriT) sequence defining a leading region sequence, a T-strand, and a T-strand complement. The method comprises inserting the isolated nucleic acid molecule into the leading region sequence, thereby generating an improved conjugative element, wherein: the isolated nucleic acid molecule is inserted such that the anti-defense genes are inserted within about 35 kb from the oriT and/or within the about 30 open reading frames (ORFs) closest to the oriT, and are encoded on the T-strand complement; and conjugation efficiency of the improved conjugative element is increased compared to conjugation efficiency of the basic conjugative element.

Definitions and embodiments mentioned above and which may be relevant to the use of the isolated nucleic acid molecule also apply here, and vice versa. Some particularly relevant embodiments may be pointed out or explicitly repeated.

In some embodiments, the present invention provides an improved conjugative element for use in a method of modulating a recipient bacteria, wherein the improved conjugative element comprises:
an origin of transfer (oriT) sequence defining a leading region sequence, a T-strand, and a T-strand complement;
at least one anti-defense gene encoded on the T-strand complement, within the leading region sequence, within about 35 kb from the oriT and/or within the about 30 open reading frames (ORFs) closest to the oriT; and
a cargo gene capable of modulating the recipient bacteria,
and the recipient bacteria expresses a defense gene product which is inhibited by a product of the at least one anti-defense gene.

Definitions and embodiments mentioned above and which may be relevant to the improved conjugative element for use also apply here, and vice versa. Some particularly relevant embodiments may be pointed out or explicitly repeated.

In some embodiments, the modulating comprises inhibiting growth of the recipient bacteria. In some embodiments, the modulating comprises enhancing growth of the recipient bacteria. In some embodiments, the modulating comprises eradicating the recipient bacteria.

In some embodiments, there is provided a method for modulating at least one feature of recipient bacteria, the method comprising contacting the recipient bacteria with donor bacteria comprising a conjugative element, which comprises:
an origin of transfer (oriT) sequence defining a leading region sequence, a T-strand, and a T-strand complement;
at least one anti-defense gene encoded on the T-strand complement, within the leading region sequence, within about 35 kb from the oriT and/or within the about 30 open reading frames (ORFs) closest to the oriT; and
a cargo gene capable of modulating the recipient bacteria,
wherein the recipient bacteria express a defense gene product which is inhibited by a product of the at least one anti-defense gene.

Definitions and embodiments mentioned above and which may be relevant to the methods also apply here, and vice versa. Some particularly relevant embodiments may be pointed out or explicitly repeated.

In some embodiments, the recipient bacteria are in a natural environment. In some embodiments, the recipient bacteria are in a human-designed environment.

The phase “at least one feature” with reference to the recipient bacteria may relate to any relevant feature which modulation thereof may resolve or ameliorate a problem. Non-limiting examples for features include the concentration of the bacteria, sensitivity/resistance of the bacteria to drugs, ability of the bacteria to secrete factors (e.g. toxins), ability of the bacteria to bind to certain structures, ability to tag or visualize bacteria, ability of bacteria to degrade or sequester contaminants or greenhouse gases, ability of bacteria to produce compounds or degrade compound for industry or pharmaceuticals, ability to isolate mining products, etc.

In some embodiments, the natural environment is a living, or organic, natural environment, such as animal body, organ, or tissue; or a plant or plant part. In some embodiments, the natural environment is a non-organic natural environment, such as soil, air, or water such as freshwater, a marine environment, a body of water, or groundwater. In some embodiments, the human-designed environments include bio-reactors, water treatment plants, water systems, hospital surfaces, medical equipment, vessels (e.g. ships), filters (e.g., percolating filters and slow sand filters), pipes, and the like.

In some embodiments, the recipient bacteria are associated with a disease, disorder, or condition of the animal or plant.

In some embodiments, the recipient bacteria belong to a phylum selected from Firmicutes, Bacteroidetes, Actinobacteria, and Proteobacteria. In some embodiments, the bacteria belong to genus selected from Prevotella, Bacteroides, Faecalibacterium, Ruminococcus, Blautia, and Clostridium.

In some embodiments, the condition, disorder, or disease are selected from dysbiotic microbiota, Clostridioides difficile infection (CDI), inflammatory bowel disease (IBD), irritable bowel syndrome (IBS), multidrug-resistant infections, obesity, metabolic syndrome, non-alcoholic fatty liver disease, neuropsychiatric diseases, diabetes mellitus and systemic autoimmune diseases.

In some embodiments, the present invention provides a method of treating or preventing a disease, disorder, or condition associated with recipient bacteria in a subject in need thereof, the method comprising administering to the subject the donor bacteria disclosed herein, wherein the recipient bacteria express a defense gene product which is inhibited by a product of the at least one anti-defense gene

Definitions and embodiments mentioned above and which may be relevant to the methods of treatment also apply here, and vice versa. Some particularly relevant embodiments may be pointed out or explicitly repeated.

In some embodiments, the donor bacteria comprise an improved conjugative element which comprises a cargo gene for treating the disease, disorder, or condition. The improved conjugative element of the invention enables the cargo gene to be transferred into the recipient bacteria by overcoming the bacterial defense systems.

The term “treating”, as used herein, refers to means of obtaining a desired physiological effect. The effect may be therapeutic in terms of partially or completely curing a disease and/or symptoms attributed to the disease. The term comprises inhibiting the disease, i.e. arresting its development; or ameliorating the disease, i.e. causing regression of the disease, e.g., by eliminating or ameliorating its symptoms.

The term “preventing”, as used herein, refers to causing a condition or symptoms thereof not to appear in the subject, or delaying the onset of such condition or symptoms, such that they do not appear at the time they are expected to appear based on similar cases, or causing the condition or symptoms to appear at a diminished level.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical objects of the article. By way of example, “an element” means one element or more than one element.

The term “about” when referring to a measurable value such as an amount, a ratio, and the like, is meant to encompass variations of +10% of the indicated value, as such variations are also suitable to perform the disclosed invention. Any numerical values appearing in the application are intended to be construed as if preceded by “about”, unless indicated otherwise.

The term “kb” or “kbp” refers to kilobases, or 1,000 bases.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims which follow.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Methods

### Datasets and Initial Annotation

The assemblies of all genomes and metagenomes from NCBI whole-genome projects (WGS) and all assembled metagenomes available from EBI Mgnify were downloaded on Mar. 14, 2020. After excluding genomes from Metazoa, Fungi, and Viridiplantae, the dataset included 596,338 genomes and 22,923 metagenomes from various ecosystems. This dataset contained more than 783 billion contigs of at least 10 kbp. In WGS, 31,119 sequences were explicitly annotated as plasmids. Gene calling and initial annotation were performed using prodigal version 3.0.0 and Prokka87 version 1.14.6.

Relaxase/traM and oriT Detection

Detection of relaxase and traM relaxosome genes was done using hmmsearch (HMMer version 3.3.2, e-value cutoff 1.00E-06) against all the proteins encoded by genomic and metagenomic sequences in our dataset. The profile HMMs were acquired from Pfam and MOBscan databases. Contigs with more than two relaxase or TraM hits were filtered out. Known oriT sequences were retrieved from oriTfinder (343 oriT sequences) and oriT-strast (112 sequences). The search for oriT sequences was performed using BLAST (BLAST+2.10.0, e-value cutoff 1.00E-06) against relaxase/traM-containing contigs (11,908 WGS plasmids and 1,019,093 genomes and metagenomes). Known oriT sequences were detected in 3,753 annotated plasmids with relaxase and in 196,414 relaxase-containing genomes and metagenomes. For contigs with more than one oriT sequence hit, only the oriT with the best BLAST score were considered. The distance between the relaxase/traM gene and the oriT was calculated as the number of nucleotides between the end of the relaxase/traM gene and the start of the oriT. oriT that were in close proximity to the relaxase/traM gene were sought. Thus, contigs in which this distance between the two was more than 3,500 bp were filtered out. Contigs in which the oriT was partially contained within the relaxase gene were included (resulting in a negative distance), but cases in which the oriT was entirely contained within the relaxase gene were excluded. Contigs in which relaxase genes or the oriT were at the ends of the contigs were also filtered out (first or last annotated sequences) since it impeded our ability to determine the relative location of the oriT and the relaxase/traM gene. Overall, this filtering process yielded 3,192 WGS annotated plasmids and 180,866 potential conjugative elements containing a relaxase/traM gene and an oriT.

### De-Duplication of Redundant Sequences

To avoid duplicate sequences, all 491,157 ORFs of the 3,192 WGS plasmid contigs containing relaxase and oriT were clustered using CD-HIT (version 4.6). According to this clustering, the percentage of shared ORFs was calculated for each pair of contigs. If two plasmids shared more than 90% of the ORFs, the plasmid with fewer ORFs was filtered out. This process yielded 1,554 representative plasmids. The same de-duplication process was performed for 180,866 potential conjugative elements identified in genomic and metagenomes sequences, yielding 17,151 non-redundant contigs of potential conjugative elements. Combining the plasmids with the rest of the potential conjugative elements resulted in a total of 18,489 non-redundant contigs of potential conjugative elements. The host phylogenetic distribution of these non-redundant contigs was mapped to the bacterial subtree of iTol46 and generated using ggtreeExtra.

### Anti-Defense and Mobility Gene Annotation

Protein families with known anti-defense functions were modeled using 120 profile HMMs. To characterize the plasmid's transfer genes, specific conjugation proteins were identified, such as type IV secretion system proteins, using HMMs downloaded from Pfam or computed based on proteins from relevant KEGG orthologs. To identify transposases, 49 HMMs from TnpPred data archive were used. Hmmsearch (with an e-value cutoff of 1.00E-06) was performed against all non-redundant potential conjugative elements sequences containing a relaxase/traM and an oriT.

### Statistical Analysis and ORF Clustering

To test which ORF positions in the leading region of plasmids were significantly enriched with anti-defense genes, a Fisher's exact test (one-sided, p-value<0.05) was performed on the anti-defense gene count at each location summing over a sliding window of five ORFs (anti-defense gene count versus the total number of genes). The same test was performed separately for each anti-defense category (namely anti-CRISPRs, anti-restriction genes, and SOS inhibitors). This analysis was performed on the 1,554 sequences of annotated plasmids for positions with at least 50 ORFs. The p-values were corrected for multiple testing using FDR (alpha=0.05). To cluster the genes in the leading region (first 30 ORFs) of all non-redundant potential conjugative sequences, MMseqs (with sensitivity of 0.75 and coverage of 0.5) were used. The ORFs in 105 gene families with more than 450 ORFs were aligned using MAFFT (version 7.475), and an HMM was constructed from each alignment. Hmmsearch (e-value cutoff 1.00E-06) of these HMMs was performed against all potential conjugative sequences. To statistically test the enrichment of each gene family in the first 30 ORFs in the leading region, a one-sided Fisher's exact test was performed, and the p-values after FDR correction were calculated (alpha=0.001). Fourteen of the 105 gene families were found not to be significantly enriched in the leading regions and were omitted from downstream analyses. Known conserved domains within the hypothetical gene families were identified using NCBI CDD and HHpred (databases: PDB, Pfam, TIGERFAMs; e-value cutoff 1.00E-10). ORFs were annotated based on significant hits to conserved domains. In gene families with ORFs that received different annotations, the most frequent annotation in the gene family was used. In each of the 91 significantly enriched gene families, the orientation of each of its ORFs relative to the oriT position was examined. The overall orientation of the gene family was defined based on the majority of its ORFs. The log-odds co-occurrence ratio of each pair of gene families was calculated as the log (base 2) of the ratio of the frequency of co-occurring gene pairs to the product of the frequencies of each gene separately.

### Frpo and ssi Promoter Identification

To identify known Frpo/ssi sequences in our anti-defense islands, a BLAST dataset of all the intergenic regions larger than 350 bp in the leading regions of all potential conjugative elements was created. A BLAST search (BLAST+2.10.0, e-value cutoff 1.00E-06) was then performed against five known Frpo/ssi sequences. New candidate Frpo sequences were detected by seeking the consensus sequences of the −35, −10 (5-TTGACA-3′ and 5′-TATAAT-3′, respectively), and the UP-element regions in the intergenic regions of the islands represented in FIG. 2. A BLAST search of the putative Frpo candidates found in these islands was then performed against all the leading regions of all potential conjugative elements. The DNA secondary structures of the Frpo/ssi elements were calculated using the RNAfold web server with the 2004 David H. Mathews model for DNA.

### Bacterial Strains and Construction of F Plasmids Variants

Escherichia coli K-12 MG1655 strain cells (used both as donor and recipient) were cultured at 30° C. or 37° C. in lysogeny broth (LB) supplemented with antibiotics at the following concentrations: Tetracycline (10 μg/ml) streptomycin (100 μg/ml), chloramphenicol (25 μg/ml), kanamycin (50 μg/ml), carbenicillin (100 μg/ml).

Gene insertion or deletion on the E. coli MG1655 K12 F-Tn10 plasmid (F+[F1−10 (Tn10)], thr-1, araC14, leuB6(Am), lacY1, glnX44(AS), galK2(Oc), galT22, λ-, ΔtrpE63, xylA5, mtl-1, thiE1, GenBank accession No. MK492260) was performed in a LY823 strain, see Table 2, using Lambda Red recombination (Yu, D. et al. An efficient recombination system for chromosome engineering in Escherichia coli. Proceedings of the National Academy of Sciences 97, 5978-5983 (2000)).

For adding the anti-defense gene acrIIa4, the Frpo-acrIIa4-Cm cassette (SEQ ID NO: 1) or yfhB_END-Frpo-acrIIa4-Cm cassette (SEQ ID NO: 2) were inserted into the F plasmid in the indicated orientations. For insertion of yfhB_END-Frpo-acrIIa4-Cm (SEQ ID NO: 2) into the leading strand in order to obtain EC207, the primers of SEQ ID No: 3 (End_yfhB_Red_F)+SEQ ID No: 4 (Red_acr4_R) were used. For insertion Frpo-acrIIa4-Cm cassette (SEQ ID NO: 1) into the lagging strand in order to obtain EC198, the primers of SEQ ID No: 5 (Red.Lag.frpo2_F)+SEQ ID No: 6 (Red.Lag.frpo2_R) were used. For insertion Frpo-acrIIa4-Cm cassette (SEQ ID NO: 1) into the leading strand in order to obtain EC199, the primers of SEQ ID No: 7 (RevRed.frpo2_F)+SEQ ID No: 8 (RevRed.frpo2_R) were used.

TABLE 1
sequences

| SEQ ID | Sequence |
|---|---|
| 1 | gtccctgtcgccggggctgatacagccacaacctg |
| | ccgcactgactgtccagggtaaatgaacagcccac |
| | aaaattttttacccaaaacccggtaaaaaattttg |
| | cgttctgcccaggacaggtgtgtcaggccgtggca |
| | gtgatgccccttgcggcgaaggttctcaaaaaggt |
| | gatggtgaataccggagggaacaaaaggcaggtcc |
| | ccgcaaaacgtcaaaaccgcggctcccgaaactca |
| | aaagatgatgggggctgaaaacatcaacagaagga |
| | gacacatcatgaacataaatgacttgatcagagag |
| | ataaagaataaagactacactgtcaagttatcggg |
| | aacggacagcaattcgatcacccagttaataatcc |
| | gggtaaataatgacggcaatgaatatgtcatttca |
| | gagtccgaaaatgaatccattgtcgaaaaattcat |
| | ttcggcgttcaagaatggctggaatcaagaatacg |
| | aagatgaggaggaattttacaacgacatgcaaaca |
| | atcacactgaaaagtgaattaaattaactcggtac |
| | caaattccagaaaagaggcctcccgaaaggggggc |
| | cttttttcgttttggtccccgacgtctgtaatgta |
| | gcctaagccctatgcccatgatctactgacgtcca |
| | ggcatttgagaagcacacggtcacactgcttccgg |
| | tagtcaataaaccggtaaaccagcaatagacataa |
| | gcggctatttaacgaccctgccctgaaccgacgac |
| | cgggtcgaatttgctttcgaatttctgccattcat |
| | ccgcttattatcacttattcaggcgtagcaaccag |
| | gcgtttaagggcaccaataactgccttaaaaaaat |
| | tacgccccgccctgccactcatcgcagtactgttg |
| | taattcattaagcattctgccgacatggaagccat |
| | cacaaacggcatgatgaacctgaatcgccagcggc |
| | atcagcaccttgtcgccttgcgtataatatttgcc |
| | catagtgaaaacgggggcgaagaagttgtccatat |
| | tggccacgtttaaatcaaaactggtgaaactcacc |
| | cagggattggctgagacgaaaaacatattctcaat |
| | aaaccctttagggaaataggccaggttttcaccgt |
| | aacacgccacatcttgcgaatatatgtgtagaaac |
| | tgccggaaatcgtcgtggtattcactccagagcga |
| | tgaaaacgtttcagtttgctcatggaaaacggtgt |
| | aacaagggtgaacactatcccatatcaccagctca |
| | ccgtctttcattgccatacggaactccggatgagc |
| | attcatcaggcgggcaagaatgtgaataaaggccg |
| | gataaaacttgtgcttatttttctttacggtcttt |
| | aaaaaggccgtaatatccagctgaacggtctggtt |
| | ataggtacattgagcaactgactgaaatgcctcaa |
| | aatgttctttacgatgccattgggatatatcaacg |
| | gtggtatatccagtgatttttttctccattttagc |
| | ttccttagctcctgaaaatctcgataactcaaaaa |
| | atacgcccggtagtgatcttatttcattatggtga |
| | aagttggaacctcttacgtgccgatca |
| 2 | aacgccgtttcgcaaaccgcgcttcacgtcgttgc |
| | ggcgggtccctgctcattgcctgccgggtgccaca |
| | cacccgtatgagtccctgtcgccggggctgataca |
| | gccacaacctgccgcactgactgtccagggtaaat |
| | gaacagcccacaaaattttttacccaaaacccggt |
| | aaaaaattttgcgttctgcccaggacaggtgtgtc |
| | aggccgtggcagtgatgccccttgcggcgaaggtt |
| | ctcaaaaaggtgatggtgaataccggagggaacaa |
| | aaggcaggtccccgcaaaacgtcaaaaccgcggct |
| | cccgaaactcaaaagatgatgggggctgaaaacat |
| | caacagaaggagacacatcatgaacataaatgact |
| | tgatcagagagataaagaataaagactacactgtc |
| | aagttatcgggaacggacagcaattcgatcaccca |
| | gttaataatccgggtaaataatgacggcaatgaat |
| | atgtcatttcagagtccgaaaatgaatccattgtc |
| | gaaaaattcatttcggcgttcaagaatggctggaa |
| | tcaagaatacgaagatgaggaggaattttacaacg |
| | acatgcaaacaatcacactgaaaagtgaattaaat |
| | taactcggtaccaaattccagaaaagaggcctccc |
| | gaaaggggggccttttttcgttttggtccccgacg |
| | tctgtaatgtagcctaagccctatgcccatgatct |
| | actgacgtccaggcatttgagaagcacacggtcac |
| | actgcttccggtagtcaataaaccggtaaaccagc |
| | aatagacataagcggctatttaacgaccctgccct |
| | gaaccgacgaccgggtcgaatttgctttcgaattt |
| | ctgccattcatccgcttattatcacttattcaggc |
| | gtagcaaccaggcgtttaagggcaccaataactgc |
| | cttaaaaaaattacgccccgccctgccactcatcg |
| | cagtactgttgtaattcattaagcattctgccgac |
| | atggaagccatcacaaacggcatgatgaacctgaa |
| | tcgccagcggcatcagcaccttgtcgccttgcgta |
| | taatatttgcccatagtgaaaacgggggcgaagaa |
| | gttgtccatattggccacgtttaaatcaaaactgg |
| | tgaaactcacccagggattggctgagacgaaaaac |
| | atattctcaataaaccctttagggaaataggccag |
| | gttttcaccgtaacacgccacatcttgcgaatata |
| | tgtgtagaaactgccggaaatcgtcgtggtattca |
| | ctccagagcgatgaaaacgtttcagtttgctcatg |
| | gaaaacggtgtaacaagggtgaacactatcccata |
| | tcaccagctcaccgtctttcattgccatacggaac |
| | tccggatgagcattcatcagggggcaagaatgtga |
| | ataaaggccggataaaacttgtgcttatttttctt |
| | tacggtctttaaaaaggccgtaatatccagctgaa |
| | cggtctggttataggtacattgagcaactgactga |
| | aatgcctcaaaatgttctttacgatgccattggga |
| | tatatcaacggtggtatatccagtgatttttttct |
| | ccattttagcttccttagctcctgaaaatctcgat |
| | aactcaaaaaatacgcccggtagtgatcttatttc |
| | attatggtgaaagttggaacctcttacgtgccgat |
| | ca |
| 3 | tcttcctcttcttcctcttcttcctcttctttccg |
| | ttcctctcctgcttaaacgccgtttcgcaaaccg |
| 4 | tttcccaggcgtcctacgaggatgaccttgttaat |
| | gccacgaactgccattgatcggcacgtaagagg |
| 5 | cttacctggtttcatcttcataccttattgggtgt |
| | attaaaaacagtagggtccctgtcgccggggct |
| 6 | ctgatgttgagcagtcatggagaattgaaacttgt |
| | ttaagactttggcagtgatcggcacgtaagaggtt |
| | ccaac |
| 7 | tcttcctcttcttcctcttcttcctcttctttccg |
| | ttcctctcctgcttatgatcggcacgtaagaggtt |
| | ccaac |
| 8 | gatgaccttgttaatgccacgaactgccatgatgt |
| | gtctccttctgttgagtccctgtcgccggggct |

Modified F plasmids were transferred to the donor strain K12 MG1655 rpsL (StrepR) (EC85) by conjugation to generate donor strains EC207, EC199, EC198, and EC196. Cloning of plasmids containing SpyCas9 to generate the recipient strains EC141 and EC142 was performed by Gibson Assembly and verified by Sanger sequencing. SpyCas9 sequence was obtained from the Addgene plasmid: #101044.

The strains generated are presented in Table 2.

TABLE 2

| Strain | Genotype |
|---|---|
| LY823 | K12 MG1655 rpsL (StrepR) / F-Tn10, parSPMT1-FRT, |
| | ΔFrpo2::FRT |
| EC85 | K12 MG1655 rpsL (StrepR) |
| EC66 | K12 MG1655 |
| EC196 | EC85 / F-Tn10, parSPMT1-FRT, ΔFrpo2::FRT |
| EC198 | EC85 / F-Tn10, parSPMT1-FRT, ΔFrpo2::FRT, |
| | Frpo2-acriiA4-Cm (NAME: Lag: Frpo2-acrIIA4) |
| EC199 | EC85 / F-Tn10, parSPMT1-FRT, ΔFrpo2::FRT, |
| | Frpo2-Cm-acriiA4 (NAME: Lead: acrIIA4-Frpo2 rev) |
| EC207 | EC85 / F-Tn10, parSPMT1-FRT, ΔFrpo2::FRT, |
| | Frpo2-acriiA4-Cm (NAME: Lead: Frpo2-acrIIA4) |
| EC141 | EC66 / SpyCas9 (gRNA targeting F-Tn10) |
| EC142 | EC66 / SpyCas9 (Non-targeting gRNA) |

### Conjugation Assays

Overnight cultures in LB and selective antibiotics of recipient and donor cells were diluted 1:100 and grown to OD₆₀₀ of 0.4. The cells were washed once with LB (2 min, 9000 rpm) and resuspended with 50 μl LB per conjugation. 30 μl of donor and 30 μl of recipient cultures were mixed in an Eppendorf tube, and 20 μl of the mix were plated on an LB-agar plate both with and without 0.1 mM arabinose, and incubated for 2 hours at 37° C. Following incubation, cells were resuspended from the agar with 1 ml 1×PBS, serially diluted, and plated on LB agar supplemented with the appropriate antibiotics to select for recipient (R) or transconjugant (T) populations (recipients were selected by Kanamycin which is encoded on the plasmids containing SpyCas9—EC141 and EC142; donor and transconjugant were selected by tetracycline encoded on the F plasmid), with and without 0.1 mM arabinose for activating SpyCas9. The conjugation frequency was quantified as T/(R+T).

### Example 1: Leading Regions of Conjugative Elements are Enriched with Anti-Defense Genes

The position of anti-defense genes in relation to the origin of transfer was examined. To this end, all sequences annotated as plasmids in the NCBI's Whole Genome Shotgun (WGS) database were analyzed. These included 31,119 sequences of plasmids and plasmid fragments, out of which 11,908 sequences contained a relaxase or traM relaxosome gene.

Relaxosome components are typically encoded in the lagging region adjacent to the origin of transfer (oriT). Thus, to detect the leading region oriT sequences were searched for in the relaxase/traM-containing plasmids. This resulted in 3,192 sequences encoding relaxosome components in close proximity to a known oriT sequence. To reduce the bias towards plasmids that are overrepresented in the databases, highly similar plasmids were removed (see Methods) and 1,554 representative plasmid sequences containing relaxase/traM close to oriT were used.

The inventors next searched for known anti-defense genes within these plasmids. These included genes encoding for anti-CRISPR proteins, which antagonize the activity of CRISPR-Cas systems; anti-restriction proteins, inhibiting restriction endonucleases; and SOS-inhibitors, which suppress the potentially deleterious host SOS-response elicited by plasmid entry. Notably, the SOS response may also induce the production of nucleases that could provoke the degradation or mutation of the transferred DNA.

The relative abundance of anti-defense proteins at each position of the plasmid sequences with respect to the location of the oriT sequence was measured. The frequency of anti-defense proteins at each position revealed that the leading region of these elements is highly enriched with anti-defense proteins (FIG. 1A). Specifically, the 30 first ORFs of the leading region were significantly enriched with anti-defense genes. Performing separate analyses for each of the anti-defense categories considered: anti-CRISPRs, anti-restriction, and SOS-inhibition, showed that the strong anti-defense enrichment in the leading region is repeated in each category (FIG. 1B).

The well-annotated plasmids in the NCBI WGS database, which were used for the initial analyses, originate from a relatively limited diversity, consisting mainly of pathogens and model organisms, and do not include ICEs. To examine how general the detected phenomenon is, putative conjugative elements, which may be unannotated plasmids or ICE-containing sequences were sought within all publicly available genomes and metagenomes from NCBI and EBI. Relaxase/TraM was searched for in proximity to an oriT sequence to identify their leading region. After excluding the well-characterized plasmids that have already been analyzed, 17,515 additional non-redundant putative conjugative elements were found. This set was scanned for anti-defense genes and, again, a very strong enrichment of anti-defense genes encoded in the leading regions of these putative conjugative elements was found (FIG. 1B and Methods). This corroborates the findings and demonstrates that, across a large variety of conjugative elements, the genes that are transferred first to the host are disproportionally involved in inhibiting the host's defense systems.

### Example 2: Uncovering Various Anti-Defense-Related Proteins in the Leading Region

To identify and characterize the most frequent gene families in the leading regions, the first 30 genes of all 18,489 non-redundant conjugative elements were clustered. Testing the enrichment of these gene families in the leading region revealed that out of the 105 largest families (with more than 450 genes each), 91 were significantly enriched in the first 30 ORFs (Fisher's exact test; p-value=0.001 after accounting for multiple testing, see Methods).

These 91 gene families included, in addition to known anti-defense genes, also three main anti-defense-related functional groups. One of the most prominent functional groups was “orphan” DNA-methyltransferases, which presumably methylates conjugative elements to protect them from the host R-M systems, as demonstrated for bacteriophages and proposed for other mobile elements, including plasmids.

ssDNA-binding proteins (SSB) were frequently found to be encoded in these regions, in most cases adjacent to SOS inhibitors (PsiA and PsiB). Several studies demonstrated the importance of plasmid-encoded SSB proteins for proper SOS-inhibition by PsiB, and their possible role in plasmids evasion from the host CRISPR-Cas systems by repairing double-strand DNA breaks. SSB proteins are also known to protect ssDNA intermediates from nuclease degradation. They directly interact with many different bacterial genome maintenance proteins, including recombination, repair, and replication proteins, such as polymerases. These suggest they might have multiple protective functions in early conjugation stages.

Toxin and antitoxins were also highly represented in the leading regions, including both toxin-antitoxin (TA) systems and “orphan” antitoxins. Although TA systems are also encoded in other regions of conjugative element genomes, their overrepresentation in the leading region suggests a potential protective role in establishing plasmids and ICEs. TA could serve either as an “addiction system” of the basic conjugative element, a defense system against other MGEs, or as an anti-defense system with antitoxins countering the function of host toxin-antitoxins, as previously shown in bacteriophages.

Notably, a major portion (~25%) of the prevalent gene families in the leading region were uncharacterized. Given the considerable overrepresentation of anti-defense genes in this region, it was expected that many of these uncharacterized families may have anti-defense-related functions. Investigating the families lacking a clear functional annotation revealed numerous putative anti-defense-related functions, including anti-restriction activity, protection against nucleases, DNA repair, and association with the SOS-inhibition mechanism (see Table 3).

The fact that the most abundant gene families within the leading region encode numerous anti-defense-related functions corroborates the findings, indicating that this region plays a key role in protection against host defense systems.

TABLE 3
Uncharacterized gene families enriched in the leading
region that have putative anti-defense functions

| | | InterPro | Putative anti-defense-related |
|---|---|---|---|
| Occurrence | DUFs^{b} | accession | function |
| 7,173 | DUF1380 | IPR009811 | klcAHS anti-restriction (0.4% of |
| | | | the gene annotations) |
| 4,235 | DUF4496 | IPR028034 | HU domain, involved in DNA |
| | | | protection and repair |
| 3,115 | — | NA | Similarity to methyltransferase. |
| 2,649 | DUF4942 | IPR031339 | Domain found in |
| | | | methyltransferases of |
| | | | R-M systems |
| 2,541 | DUF905 | IPR009253 | Consistently adjacent to |
| | | | SOS-inhibitors |
| 731 | — | | ccgAII anti-restriction and |
| | | | putative regulator of RecA |

^{a}Based on the size of the gene family.
^{b}Presence of domains of unknown functions; DUF: domain of unknow function.
Occur: occurrences (number of genes in the family)

### Example 3: Anti-Defense Islands in the Leading Regions of Conjugative Elements

Examining the leading regions of conjugative elements revealed that the anti-defense genes tend to cluster into islands, as was previously reported for MGEs that contain clustered anti-defense genes. These “anti-defense islands” include different combinations of anti-defense and anti-defense-related genes adjacent to each other. For example, such an island was located in the leading region of a conjugative element of Salmonella enterica that contains two anti-CRISPR genes (acrIC6, and acrIF16) in close proximity to gene encoding for anti-restriction (klcAHS), SOS-inhibitors (psiA and psiB), methyltransferases, ssDNA-binding proteins, and a TA system (higB-higA, FIG. 2). A similar island, with a few differences, was identified in the leading region of a plasmid from Serratia marcescens. This island harbored an anti-CRISPR gene inhibiting a different type of CRISPR-Cas system (acrIE9), and an additional antitoxin gene (hipB, FIG. 2). The hipB antitoxin gene is usually located as part of hipBA operons and counters the toxicity of HipA¹⁷. However, in this anti-defense island, it was found next to a higA/relE TA system and may function as an “orphan” antitoxin inhibiting host TA defense systems.

Many of the anti-defense islands were flanked, in the oriT-distal region, by an operon of umu genes or homologs thereof, which essentially forms the terminating boundary of the island. These genes are plasmids-encoded homologs of umuC and umuD, which form chromosomal translesion DNA synthesis polymerases (DNA polymerase V). While they are highly abundant in the leading region, they are not encoded in the orientation that would allow their expression from the ssDNA first transferred to the recipient bacteria (in 99.6% of the islands). Therefore, they are not expected to be expressed early upon conjugation. One case of a large anti-defense island (from a conjugative element recovered insect metagenomics) appeared to consist of two adjacent islands separated by a transposase. An operon of the umu genes flanks each of these two adjacent islands (FIG. 2).

Supplementing the search with anti-defense islands that included primarily of uncharacterized gene families led to the detection of several additional anti-defense islands that could not have been detected based on the initial dataset of known anti-defense systems. One of the interesting islands found owing to the uncharacterized gene enriched in the leading regions was an anti-defense island in a conjugative element from Streptococcus pneumoniae. These bacteria can spread a pneumococcal disease in immunocompromised individuals and, in severe cases, can cause hearing loss, brain damage, and death. This S. pneumoniae conjugative element also harbors antibiotic-resistance genes against several antimicrobials, including tetracycline and chloramphenicol. The anti-defense island in the leading region of this element included a unique combination of anti-defense genes: a methyltransferase, two infrequent anti-CRISPRs (acrIB and acrIIA21), two anti-restriction proteins (darB), a TA system (abiEii-abiEi), and two uncharacterized gene families prevalent in leading regions, in close proximity to a spxA gene (FIG. 2). SpxA is a transcriptional regulator involved in repressing the X-state (competence induction) in S. pneumoniae, a general stress response mechanism in this species. The activation of this system leads to the expression of more than 100 genes, representing a significant burden for the cell. The plasmid-encoded SpxA may serve as an “anti-X-state” protein that prevents the stress response following the entry of foreign DNA.

### Example 4: Single-Stranded DNA Promoters are Widespread in the Anti-Defense Islands

Examining the orientation of the 91 gene families enriched in the leading region, it was found that all the anti-defense and anti-defense related functions were coded on the same strand relative to the oriT. Specifically, the leading region anti-defense genes were consistently found on the T-strand complement, such that they could be transcribed from the single strand that is first transferred to the recipient, even before the plasmid's complementary strand is synthesized.

Transcription from ssDNA can be achieved using specific promoters, known as Frpo or ssi sequences, that create secondary DNA structures mimicking dsDNA, which enable recognition by RNA polymerase. Known Frpo/ssi sequences were sought in the leading regions of the 18,489 putative conjugative elements. 11,840 Frpo-type homologous promoters in 5,341 conjugative elements were detected. In each of the leading regions of the S. marcescens and the S. enterica plasmids, one Frpo-type homologous sequence was identified immediately upstream to the gene encoding the SSB protein, which is followed by SOS-inhibition genes (FIG. 2). Notably, the transcription by Frpo promoters is highly stimulated by SSB.

In two other islands (from an insect metagenomic sample and from S. pneumoniae, FIG. 2), no sequences homologous to Frpo were found. It was postulated that additional ssDNA promoters might be present in the leading region to allow early expression of island genes. After searching for novel Frpo-type sequences in other regions upstream of ORFs in the islands, Frpo-type candidates were detected based on their secondary structure and their conformance with known Frpo sequences and consensus sequences of the −35 and −10 elements (5′-TTGACA-3′ and 5′-TATAAT-3′, respectively). In the S. marcescens anti-defense island, three putative Frpo-type candidates were detected (Frpo′). In the S. enterica island, three sequences bearing distant similarity to Frpo-type sequences were identified (Frpo*), with secondary structures similar to known Frpos sequences but considerable differences in the conserved −35 and −10 elements. Analyzing the islands from insect metagenome led to the detection of three additional Frpo-type candidates (Frpo′) and four putative Frpo candidates with only distant similarity (Frpo*) to known Frpo sequences.

The putative Frpo-type candidates found in the above-mentioned islands were searched for within the leading region of all 18,489 conjugative elements. 5,768 additional Frpo′ and 736 Frpo* candidates were identified, presenting high and limited similarity to Frpo sequences correspondingly. Overall, the analysis of the regions upstream to ORFs in the islands revealed new and putative Frpo-type promoters in the leading regions of numerous conjugative elements. These results indicate that Frpo promoters are widespread in anti-defense islands and can potentially allow early expression from ssDNA during the very early stages of conjugation as part of the establishment phase in the host, and specifically for evading the host defense systems.

### Example 5: Enhancing Conjugation Efficiency: Optimizing the Inhibition Effect of Anti-CRISPR Genes Under Frpo Promoter Expression in the Leading Region of the Plasmid

The effect of positioning of anti-defense genes in conjugating plasmids on the conjugation efficiency in the presence of a defense system within the recipient bacteria was examined. More specifically, effects of placing an anti-defense gene under Frpo promoter control, in the leading region or the lagging region of the plasmid, and in both orientations, was tested. To achieve this, an anti-CRISPR gene (acrIIA4) was integrated in different locations and orientations into an F plasmid in the donor cell, having a genotype of K12 MG1655 rpsL (StrepR)(EC85): a) acrIIA4 gene directly downstream of the native Frpo promoter in the leading region and encoded on the T-strand complement (Lead: Frpo-acrIIA4, EC207); b) acrIIA4 gene directly downstream of an Frpo promoter in the lagging region (Lag: Frpo-acrIIA4, EC198); and c) acrIIA4 gene in the leading region and encoded on the T-strand, i.e., in reverse orientation to EC207 (Lead: acrIIA4-Frpo rev, EC199). An F plasmid not including acrIIA4 was used as a negative control (EC196). The distance of the Frpo-acrIIA4 from the oriT was 9 ORFs (~7 kb).

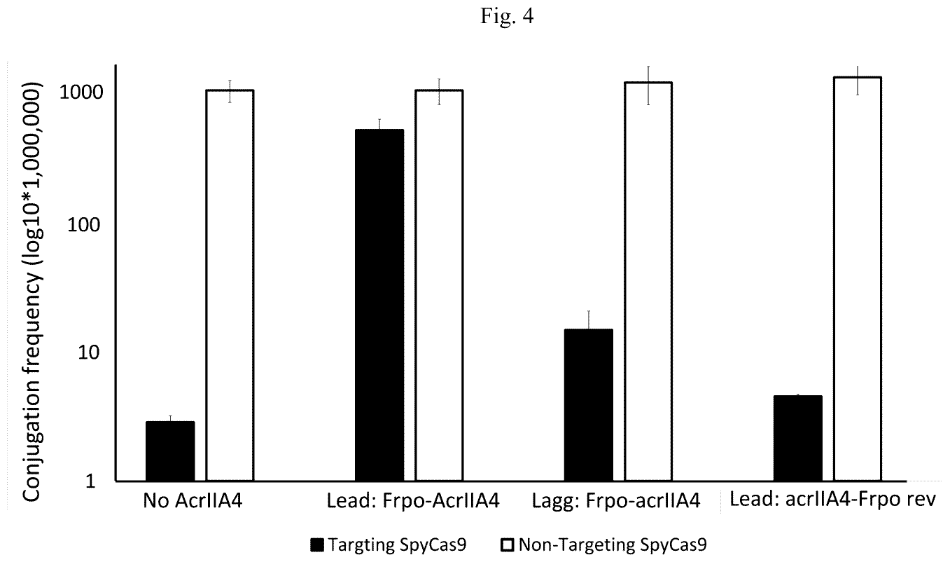

The conjugation experiments conducted included the donor variants noted above, and recipient cells containing active SpyCas9 that targets the conjugative F plasmid (EC141). The results demonstrated that placing the anti-CRISPR gene in the leading region, encoded on the T-strand complement under a single-stranded promoter (Lead: Frpo-AcrIIA4) allows for effective inhibition of the SpyCas9 CRISPR-Cas system, leading to approximately 100-5,000 fold higher conjugation frequency compared to different configurations (see FIG. 4). As a conjugation control, the same experiments were conducted with recipient cells containing non-targeting SpyCas9 (EC142), and found that conjugation frequency was not affected, indicating no significant differences in efficacy across the different donor variants.

### Example 6: The Effect of the Composition of the Leading Region on Conjugation Efficiency Strains and Plasmids

For conjugation experiments, E. coli K1037 is used as a donor cell, E. coli MG1655 as a recipient, and N3 native conjugative plasmid (pN3, GenBank accession FR850039.1), RP4 plasmid, pTA-Mob (Soltysiak et al., Int. J. Mol. Sci. 2019, 20, 5212, based on the RK2 plasmid conjugative machinery), and F-plasmid, and variants thereof are used as the plasmids for testing conjugation efficiency.

The leading region of pN3 harbors an anti-defense island of ~6 kb, which encodes the anti-restriction gene ardB, an inhibitor of type I restriction-modification (R-M) systems. The recipient cell MG1655 contains an active type I R-M EcoKI enzyme.

Accordingly, pN3 variants are designed to test the effect on conjugation efficiency of deleting the island, changing its position/orientation, and adding elements to the plasmid, on conjugation efficiency, while RP4, pTA-Mob, and F-plasmid variants are designed to test the effect of adding anti-defense genes with a known function in various positions/orientations on conjugation efficiency.

The following constructs are prepared by the Gibson assembly or by the lambda red recombination method:

### Pn3 Constructs:

the anti-defense island deleted;
the anti-defense island in reversed orientation;
the anti-defense island in the lagging region;
with the addition of EcoKI sites: two EcoKI recognition sites are added to the native pN3 and the variants described above to test the inhibitory effect of the ardB anti-restriction gene of pN3 in the recipient MG1655 containing an active type I R-M EcoKI enzyme; and
with an added anti-CRISPR gene in either a forward or a reverse orientation in the anti-defense island.

### RP4/pTA-Mob/F-Plasmid Constructs:

with the addition of an anti-CRISPR gene in the leading region with an ssDNA promoter, in either a forward or a reverse orientation with reference to the oriT of the plasmid.
with the addition of an anti-CRISPR gene in the leading region without an ssDNA promoter.
with the addition of an anti-CRISPR gene in the lagging region.
with the addition of one or more anti-defense or anti-defense-related gene such as anti-restriction gene(s), SOS-inhibitor(s) and/or methyltransferase(s) in the leading region, in either a forward or a reverse orientation with reference to the oriT of the plasmid.

Experiments were done as described in the methods and in Example 5 above.

For testing the effect of the anti-CRISPR gene on conjugation efficiency, the recipient bacteria are also transfected with a plasmid carrying an active CRISPR/Cas system including a spacer targeting the relevant plasmid. For an additional control, conjugation efficiency is also tested in bacteria without the CRISPR/Cas system.

Conjugation of the following plasmids is tested in bacteria with and without CRISPR/Cas system: (1) plasmids carrying no anti-CRISPR genes, (2) plasmids carrying an anti-CRISPR gene encoded on the leading strand, (3) plasmids carrying an anti-CRISPR gene encoded on the complement strand.

For testing the effect of anti-defense genes on conjugation efficiency, the recipient bacteria contain an active defense system corresponding to the anti-defense genes carried by the respective plasmid, such as restriction-modification and/or SOS-response systems.

Conjugation of the following plasmids is tested in bacteria with and without the defense system: (1) plasmids carrying no anti-defense genes, (2) plasmids carrying anti-defense genes encoded on the leading strand, (3) plasmids carrying anti-defense genes encoded on the complement strand. (4) plasmids carrying anti-defense genes with ssDNA promoter (Frpo/ssi), (5) plasmids carrying anti-defense genes without ssDNA promoter (Frpo/ssi).

1-36. (canceled)

37. A method for increasing conjugation efficiency of a basic conjugative element which comprises an origin of transfer (oriT) sequence defining a leading region sequence, a T-strand, and a T-strand complement,
the method comprising inserting into the leading region sequence of the basic conjugative element an anti-defense nucleotide sequence comprising a sequence encoding at least one anti-defense gene, thereby generating an improved conjugative element,
wherein:
following the inserting, the at least one anti-defense gene is encoded on the T-strand complement within about 35 kb from the oriT and/or within about 30 open reading frames (ORFs) closest to the oriT; and
the conjugation efficiency of the improved conjugative element is increased compared to the conjugation efficiency of the basic conjugative element.

38. The method of claim 37, wherein the at least one anti-defense gene is an anti-CRISPR gene, an anti-restriction gene, and/or an SOS inhibitor gene.

39. The method of claim 38, wherein the anti-CRISPR gene is selected from the group consisting of acrIIA4, acrIC6, acrIF16, acrIE9, acrIB, and acrIIA21; the anti-restriction gene is selected from ocr, ardA, ardB, klcAHS, and darB; and/or the SOS inhibitor is selected from psiA and psiB.

40. The method of claim 37, wherein the anti-defense nucleotide sequence further comprises a sequence encoding at least one anti-defense-related gene selected from the group consisting of a methyltransferase, a single-strand DNA-binding protein (SSB), a toxin, and an antitoxin, and following the inserting the at least one anti-defense-related gene is encoded on the T-strand complement within about 35 kb from the oriT and/or within about 30 open reading frames (ORFs) closest to the oriT.

41. The method of claim 37, wherein the anti-defense nucleotide sequence further comprises a sequence encoding at least one umu gene or a homolog thereof selected from the group consisting of a homolog of umuC and a homolog of umuD, which is positioned farther away from the oriT compared to the at least one anti-defense gene.

42. The method of claim 37, wherein the basic conjugative element is selected from a conjugative plasmid, an integrative conjugative element (ICE), and a mobile genetic element (MGE).

43. The method of claim 37, wherein the basic conjugative element does not comprise a sequence encoding anti-defense genes or anti-defense-related genes in the leading region sequence, within about 35 kb from the oriT sequence and/or within about 30 ORFs closest to the oriT.

44. The method of claim 37, wherein the basic conjugative element further comprises a sequence encoding a relaxase gene.

45. The method of claim 37, wherein the anti-defense nucleotide sequence further comprises a sequence including at least one single stranded DNA promoter selected from the group consisting of Frpo, ssiD, ssiE, ssi2, and ssi3, capable of initiating RNA transcription from single strand DNA.

46. The method of claim 37, wherein following insertion of the anti-defense nucleotide sequence, the conjugation efficiency of the improved conjugative element is increased by at least 10%, 15%, 20%, 25%, 30%, 25%, 40%, 45%, 50%, 60%, 70%, 80%, or 100% compared to the conjugation efficiency of the basic conjugative element.

47. The method of claim 37, wherein the basic conjugative element further comprises a cargo gene for transfer by conjugation, or the method further comprises inserting into the basic conjugative element or into the improved conjugative element a cargo gene for transfer by conjugation.

48. The method of claim 37, further comprising a step of transferring the improved conjugative element into donor bacterial cells.

49. The method of claim 48, wherein the donor bacterial cells are Escherichia coli cells.

50. The method of claim 48, further comprising a step of contacting recipient bacteria with the donor bacterial cells.

51. The method of claim 50, wherein the recipient bacteria express a defense gene product which is inhibited by a product of the at least one anti-defense gene.

52. A donor bacterial cell prepared by the method of claim 48.

53. A method for modulating recipient bacteria, the method comprising contacting the recipient bacteria with donor bacteria comprising a conjugative element, which comprises:
an origin of transfer (oriT) sequence defining a leading region sequence, a T-strand, and a T-strand complement;
at least one anti-defense gene encoded on the T-strand complement, within the leading region sequence, within about 35 kb from the oriT and/or within about 30 open reading frames (ORFs) closest to the oriT; and
a cargo gene capable of modulating the recipient bacteria,
wherein the recipient bacteria express a defense gene product which is inhibited by a product of the at least one anti-defense gene.

54. The method of claim 53, wherein the recipient bacteria are in a natural environment.

55. The method of claim 54, wherein the natural environment is an animal body, organ, or tissue.

56. The method of claim 54, wherein the natural environment is soil, freshwater, groundwater, a water body, or a water system.
